SEQUENCE LISTING (38 sequences)

SEQ ID NO: 1 - DNA, 18 bp, synthetic construct
  source 1..18: mol_type = other DNA; note = Forward primer FOXA2
gggagcggtgaagatgga

SEQ ID NO: 2 - DNA, 22 bp, synthetic construct
  source 1..22: mol_type = other DNA; note = Reverse primer FOXA2
tcatgttgctcacggaggagta

SEQ ID NO: 3 - DNA, 19 bp, synthetic construct
  source 1..19: mol_type = other DNA; note = Forward primer SOX17
gtggaccgcacggaatttg

SEQ ID NO: 4 - DNA, 23 bp, synthetic construct
  source 1..23: mol_type = other DNA; note = Reverse primer SOX17
cacgtcaggatagttgcagtaat

SEQ ID NO: 5 - DNA, 19 bp, synthetic construct
  source 1..19: mol_type = other DNA; note = Forward primer exNXK2-1
tgtcctgctccaccttgct

SEQ ID NO: 6 - DNA, 20 bp, synthetic construct
  source 1..20: mol_type = other DNA; note = Reverse primer exNXK2-1
cgcacaccggccttattcca

SEQ ID NO: 7 - DNA, 20 bp, synthetic construct
  source 1..20: mol_type = other DNA; note = Forward primer exPAX8
cctcggtgcacatgctttac

SEQ ID NO: 8 - DNA, 21 bp, synthetic construct
  source 1..21: mol_type = other DNA; note = Reverse primer exPAX8
gaggtctgccattcacaaagg

SEQ ID NO: 9 - DNA, 17 bp, synthetic construct
  source 1..17: mol_type = other DNA; note = Forward primer NKX2-1
gcttccccgccatctcc

SEQ ID NO: 10 - DNA, 20 bp, synthetic construct
  source 1..20: mol_type = other DNA; note = Reverse primer NKX2-1
gccatgttcttgctcagctc

SEQ ID NO: 11 - DNA, 19 bp, synthetic construct
  source 1..19: mol_type = other DNA; note = Forward primer PAX8
cgagcgactccccggcgat

SEQ ID NO: 12 - DNA, 18 bp, synthetic construct
  source 1..18: mol_type = other DNA; note = Reverse primer PAX8
gaggtctgccattcacaa

SEQ ID NO: 13 - DNA, 20 bp, synthetic construct
  source 1..20: mol_type = other DNA; note = Forward primer FOXE1
gcgacaaccccaaaaagtgg

SEQ ID NO: 14 - DNA, 17 bp, synthetic construct
  source 1..17: mol_type = other DNA; note = Reverse primer FOXE1
gcccagtagcccttacc

SEQ ID NO: 15 - DNA, 22 bp, synthetic construct
  source 1..22: mol_type = other DNA; note = Forward primer HHEX
ggacggtgaacgactacacgca

SEQ ID NO: 16 - DNA, 21 bp, synthetic construct
  source 1..21: mol_type = other DNA; note = Reverse primer HHEX
ccagacgcttcctctcggcgc

SEQ ID NO: 17 - DNA, 21 bp, synthetic construct
  source 1..21: mol_type = other DNA; note = Forward primer TG
agacacctcctacctccctca

SEQ ID NO: 18 - DNA, 20 bp, synthetic construct
  source 1..20: mol_type = other DNA; note = Reverse primer TG
tccttggacatcgctttggc

SEQ ID NO: 19 - DNA, 21 bp, synthetic construct
  source 1..21: mol_type = other DNA; note = Forward primer TSHR
tgacctttcttacccaagcca

SEQ ID NO: 20 - DNA, 23 bp, synthetic construct
  source 1..23: mol_type = other DNA; note = Reverse primer TSHR
tgctctcaaggacttacacatca

SEQ ID NO: 21 - DNA, 19 bp, synthetic construct
  source 1..19: mol_type = other DNA; note = Forward primer TPO
ctgtcacgctggttatggc

SEQ ID NO: 22 - DNA, 22 bp, synthetic construct
  source 1..22: mol_type = other DNA; note = Reverse primer TPO
gctagagacacgagactcctca

SEQ ID NO: 23 - DNA, 21 bp, synthetic construct
  source 1..21: mol_type = other DNA; note = Forward primer NIS/SLC5A5
atcgctatggcctcaagttcc

SEQ ID NO: 24 - DNA, 20 bp, synthetic construct
  source 1..20: mol_type = other DNA; note = Reverse primer NIS/SLC5A5
tccaggtactcgtaggtgct

SEQ ID NO: 25 - DNA, 21 bp, synthetic construct
  source 1..21: mol_type = other DNA; note = Forward primer GAPDH
ctataaattgagcccgcagcc

SEQ ID NO: 26 - DNA, 21 bp, synthetic construct
  source 1..21: mol_type = other DNA; note = Reverse primer GAPDH
tacgaccaaatccgttgactc

SEQ ID NO: 27 - DNA, 21 bp, synthetic construct
  source 1..21: mol_type = other DNA; note = Forward primer TP63
ctcgtccaccagtccctataa

SEQ ID NO: 28 - DNA, 21 bp, synthetic construct
  source 1..21: mol_type = other DNA; note = Reverse primer TP63
tgatggagagagagcatcgaa

SEQ ID NO: 29 - DNA, 20 bp, synthetic construct
  source 1..20: mol_type = other DNA; note = Forward primer FOXJ1
ggcataagcgcaaacagccg

SEQ ID NO: 30 - DNA, 23 bp, synthetic construct
  source 1..23: mol_type = other DNA; note = Reverse primer FOXJ1
tcgaagatggcctcccagtcaaa

SEQ ID NO: 31 - DNA, 20 bp, synthetic construct
  source 1..20: mol_type = other DNA; note = Forward primer SCGB3a2
gaggggctaaggaagtgtgt

SEQ ID NO: 32 - DNA, 20 bp, synthetic construct
  source 1..20: mol_type = other DNA; note = Reverse primer SCGB3a2
ccatccacctccgctcttta

SEQ ID NO: 33 - DNA, 20 bp, synthetic construct
  source 1..20: mol_type = other DNA; note = Forward primer AGER
gccactggtgctgaagtgta

SEQ ID NO: 34 - DNA, 20 bp, synthetic construct
  source 1..20: mol_type = other DNA; note = Reverse primer AGER
gtccggcctgtgttcagttt

SEQ ID NO: 35 - DNA, 25 bp, synthetic construct
  source 1..25: mol_type = other DNA; note = Forward primer LAMP3
tgaccactttggaaatgtggatgag

SEQ ID NO: 36 - DNA, 20 bp, synthetic construct
  source 1..20: mol_type = other DNA; note = Reverse primer LAMP3
gatggccccaatcacaggaa

SEQ ID NO: 37 - DNA, 17 bp, synthetic construct
  source 1..17: mol_type = other DNA; note = forward primer Dio1
atggccaggaacccccg

SEQ ID NO: 38 - DNA, 20 bp, synthetic construct
  source 1..20: mol_type = other DNA; note = reverse primer Dio1
gcctgctgccttgaatgaaa

(19) United States
(12) Patent Application Publication
(10) Pub. No.: US 2025/0019649 A1
(43) Pub. Date: Jan. 16, 2025

(54) GENERATION OF THYROID AND LUNG ORGANOIDS FROM STEM CELLS

(71) Applicant: Université Libre de Bruxelles, Brussels (BE)

(72) Inventors: Sabine, Aline, Nadine COSTAGLIOLA, Molenbeek Saint-Jean (BE); Mirian ROMITTI, Ixelles (BE)

(73) Assignee: Université Libre de Bruxelles, Brussels (BE)

(21) Appl. No.: 18/714,282

(22) PCT Filed: Nov. 30, 2022
(86) PCT No.: PCT/EP2022/083777

§ 371 Date: May 29, 2024

(30) Foreign Application Priority Data
Nov. 30, 2021 (EP) 21211355.9

(51) Int. Cl. C12N 5/071; A61K 35/42; A61K 35/55

(52) U.S. Cl. CPC C12N 5/0617; A61K 35/42; A61K 35/55; C12N 5/0688; C12N 2501/01; C12N 2501/15; C12N 2501/16; C12N 2501/375; C12N 2501/39; C12N 2503/04; C12N 2506/02; C12N 2513/00

(57) ABSTRACT

The invention relates to a method for differentiating pluripotent stem cells into thyroid or lung organoids by forming embryoid bodies, differentiating the embryoid bodies into definitive endoderm, inducing (over) expression of NKX2-1 and PAX8, and treatment with respectively, a cyclic monophosphate, a glucocorticoid, thyroid-stimulating hormone and a TGF-β inhibitor, or with a cyclic monophosphate, insulin or IGF-1, and a TGF-β inhibitor. The invention also relates to thyroid and lung organoids obtained by the methods as well as uses thereof for therapeutic applications and medicinal applications such as drug screening or research purposes.

### FIELD OF THE INVENTION

The invention belongs to the technical field of directed cell differentiation and biomedicine, and relates to methods for differentiating human stem cells into thyroid organoids or lung organoids. The invention also encompasses so-obtained thyroid organoids and lung organoids, and uses of said thyroid organoids and lung organoids, for example, for medicinal applications such as regenerative medicine, drug screening and/or research purposes.

### BACKGROUND OF THE INVENTION

Hypothyroidism is a very common disorder with a prevalence of 1% to 5% worldwide. It results from insufficient thyroid hormone (TH) production due to autoimmune damage to the thyroid gland, iodide excess or deficiency, external irradiation, genetic defects or other defects manifesting at birth (congenital hypothyroidism, CH) and surgical or radioactive thyroid ablation to treat hyperthyroidism or thyroid cancer. Despite well-established TH replacement therapy, it is estimated that up to one-third of patients do not receive an adequate treatment while a large proportion have impaired health-related quality of life, particularly psychological well-being. In addition, studies have shown that children with CH can develop motor, cognitive, and social dysfunction even when diagnosed through newborn screening followed by early institution of TH replacement. Indeed, constant exogenous supply of TH does not provide for changes in TH requirement associated with growth, puberty, pregnancy and stress. New therapeutic approaches, such as regenerative medicine, would accommodate the variation in TH demand.

Lung organoids have significant potential in the search for new treatments for almost every lung disease including, for example, lung cancer, idiopathic pulmonary fibrosis (IPF), cystic fibrosis (CF), asthma, and lung infectious diseases such as COVID-19-associated pneumonia. Lung organoids have become important tools for researchers to more closely model these human diseases. In addition, lung organoids can address questions in lung biology, such as in vivo pathways regulating lung development and mechanisms by which endogenous lung progenitors effect repair, and how these might be enhanced by small molecules or drugs.

Breakthroughs in 3-dimensional (3D) organoid cultures for many organ systems have led to new physiologically complex in vitro models to study human development and disease. Organoids are a type of 3D cell clusters that are close to the physiological conditions in the body, have self-renewal and self-organization capabilities, and have corresponding tissue and organ functions.

Human embryonic stem cell (ESC)-based protocols have been developed and led to the generation of several types of human organoids that include brain, intestine, stomach, liver, kidney, lung, endometrium, prostate, pancreas, and retina.

With regard to the thyroid, murine ESC-derived organoids have been shown to recapitulate in vitro the developmental stages of the thyroid gland with the ability to produce TH in vitro and in vivo after transplantation to mice with ablated thyroid glands. In contrast, human thyroid cells so far generated from stem cells have not shown full maturation in vitro and ability to compensate for low thyroid hormone levels when transplanted into animals devoid of thyroid tissue. These difficulties in producing functional human thyroid follicles capable of restoring thyroid function in vivo have been partially overcome by using organoids generated from suspensions of adult human thyroid cells, but with some limitations. For example, Ogundipe et al. (2021. Stem Cell Reports 16:913-924) generated thyrospheres from human thyroid glands, but 26 weeks are required to detect human thyroid tissue when these organoids are transplanted into hypothyroid mice, and plasma levels of thyroxine (T4) did not increase significantly.

Lung organoids derived from human stem cells usually show airway or alveolar characteristics and are thus not representative for the complete lung, but only for isolated compartments. For example, WO 2016/083613 describes lung organoids derived from adult stem cells that reside in the lung. These adult stem cell-derived lung organoids consist exclusively of epithelial cells and may therefore be less suitable as model of the native lung since not all cell-types responsible for lung organization and functioning are present. Miller et al. (2019 Nat Protoc 14:518-540) describes the generation of most lung cell types starting from hESC, but the protocol involves several distinct steps and generates immature cells that require transplantation in vivo to completely maturate. A preprint article by Tindle et al. (2021. bioRxiv, eLife doi: 10.7554/eLife.66417) describes the generation of an adult stem cell-derived complete lung organoid containing both proximal and distal airway epithelia. Though, the exact cellular proportion was not assessed. Taking in account that the generation of multicellular lung organoids from hESC is still challenging due to complexity and timing; and that using adult lung tissue as start material does not recapitulate early stages of lung development, there remains a need for a model that allows to study lung developmental processes and to model lung diseases.

Most attempts to derive organoids from pluripotent stem cells rely on in vitro recapitulation of known in vivo embryonic developmental signals. This approach was however not straightforward for lung organoids since the minimal pathways regulating in vivo lung lineage specification as well as their evolutionary conservation were controversial.

Further complicating thyroid and lung organoid generation from pluripotent stem cells is that lung and thyroid epithelial lineages both originate from NKX2-1⁺ foregut progenitors. Consequently, in vitro-directed differentiation of pluripotent stem cells into lung lineages has resulted in the derivation of mixed populations of lung and thyroid lineages. Serra et al. (2017 Development 144:3879-3893) revealed the key minimal signalling pathways (Wnt+BMP versus BMP+FGF) that regulate distinct lung- versus thyroid-lineage specification from foregut endoderm. They succeeded to isolate lung or thyroid progenitor pools specified from pluripotent stem cells with these minimal pathways by cell sorting, which were competent to form lung or thyroid epithelial spheres, respectively. However, the isolation step remains a hurdle for a simple method to generate lung organoids from pluripotent stem cells.

Hence, there remains a need in the art to establish such model systems, not in the least to further elucidate the molecular mechanisms underlying thyroid and lung development and function as well as dysfunction, as well as for use in therapeutic applications.

### SUMMARY OF THE INVENTION

The aspects and embodiments of the present invention address at least some, e.g., one or more, of the above discussed needs of the art.

The present inventors used forward programming by transient overexpression of NK2 homeobox 1 (NKX2-1) protein and a paired box 8 (PAX8) protein and manipulation of signalling pathways in combination with stepwise transcriptomic characterization, to generate a functional human thyroid from human pluripotent stem cells, in particular human embryonic stem cells, that recapitulates thyroid function in vitro and in vivo.

Indeed, it was shown that by manipulating developmental signalling pathways hESCs generate definitive endoderm. The inducible co-expression of NKX2-1 and PAX8 committed the definitive endodermal cells into lung and thyroid fate. Their subsequent treatment with cyclic monophosphate, a glucocorticoid (e.g. dexamethasone), a thyroid-stimulating hormone (e.g. rhTSH) and a TGF-β inhibitor lead the cells to form fully functional thyroid follicles.

As shown in the experimental section, the present inventors surprisingly found that subsequent treatment of the thyroid and lung progenitor cells with cyclic monophosphate, and insulin or insulin-like growth factor 1 (IGF-1), lead the cells to form lung organoids instead. The lung organoids were shown to comprise both epithelial and mesenchymal compartments of the lung, organized with structural features similar to the native lung. In particular, the lung organoids were shown to possess upper airway-like epithelium with basal cells and immature ciliated cells surrounded by mesodermal cells, as well as an alveolar-like domain with appropriate cell types.

In particular, the invention relates to a (in vitro or ex vivo) method for generating a thyroid organoid or a lung organoid from human pluripotent stem cells, said method comprising:
a. generating an embryoid body from human pluripotent stem cells;
b. culturing the embryoid body in a 3D matrix under conditions to induce differentiation into a definitive endoderm;
c. transiently overexpressing NKX2-1 and PAX8 in the definitive endoderm to induce differentiation of endodermal cells into thyroid and lung progenitor cells; and
d. culturing the thyroid and lung progenitor cells in the 3D matrix in a differentiation medium for a period of time sufficient to generate a thyroid organoid or a lung organoid.
   Preferably, the methods described herein are in vitro or ex vivo methods, wherein all steps are performed in vitro or ex vivo, or wherein none of the steps is performed in vivo.

The invention further relates to thyroid organoids and lung organoid obtainable by the methods of the invention and uses thereof.

The above and further aspects and preferred embodiments of the invention are described in the following sections and in the appended claims. The subject matter of appended claims is hereby specifically incorporated in this specification.

### BRIEF DESCRIPTION OF FIGURES

FIG. 1: Schematic representation of NKX2-1-PAX8 tetracycline-inducible hESC line. The coding sequences of the NKX2-1 and PAX8 genes, separated by an internal ribosome entry site (IRES) sequence, were cloned into the pInducer20 lentiviral vector backbone, which contains the sequences for the TRE/rtTA-inducible system.

FIG. 2: Schematic representation of a method according to an embodiment of the invention leading to thyroid follicle differentiation from human ESCs.

FIG. 3: Early differentiation of the NKX2-1-PAX8 tetracycline-inducible human ESC line (a) FOXA2 and SOX17 mRNA levels after Activin A (AA) treatment (day 5 of the method shown in FIG. 2). (b) Quantitative reverse transcription polymerase chain reaction (qRT-PCR) analysis for exogenous and endogenous NKX2-1 and PAX8, FOXE1, TG and TSHR after doxycycline (Dox) stimulation. (c) Gene expression curve of endogenous PAX8 and exogenous NKX2-1 and PAX8 from day 9 to day 16 of the differentiation method shown in FIG. 2.

FIG. 4: Transient overexpression of NKX2-1 and PAX8 promotes differentiation of human ESCs into thyroid follicular cells. (a) Quantification by flow cytometry of the proportion of NKX2-1^{GFP+} cells during the differentiation method as shown in FIG. 2. (b) Heatmap of normalized bulk RNA-Seq expression of thyroid genes in NKX2-1^{GFP+} cells at different stages of the thyroid differentiation method described herein. Rows represent markers and columns represent specific time points corresponding to the method shown in FIG. 2. Greyscale values in the heatmap represent mean expression levels. (c) Heatmap showing normalized expression of selected marker genes with rows representing cell clusters, while columns represent genes. The intensity of the greyscale in each square indicates the mean expression within the cluster. (d) Expression trends of thyroid genes along the pseudotime trajectory.

FIG. 5: Characterization of the proliferation and early differentiation stages of the differentiation method as shown in FIG. 2. (a) Proportion of NKX2-1^{GFP+} cells expressing the proliferation marker KI67 during the differentiation method. (b) NKX2-1, TG and TSHR gene expression curve (bulk RNA-Seq data) at different stages of the differentiation method as indicated. (c) qRT-PCR analysis of PAX8, TSHR, NIS/SLC5A5, TG and TPO genes at day 45 compared to −Dox control. ****p<0.0001 (d) Heatmap of bulk RNA Seq expression of transforming growth factor beta (TGFβ) signalling markers within NKX2-1^{GFP+} cells by differentiation.

FIG. 6: scRNA-Seq clusters characterization and CellPhone-DB heterotypic interaction between thyroid populations and other cells at day 45. (a) Violin plots showing expression levels of key thyroid markers between thyroid clusters and other cells. (b) Diagram showing selected ligand-receptor interactions using CellPhoneDB on the single-cell dataset of human thyroid organoids; P values are indicated by circle size. The grayscale shows the Log2 mean values of the average expression level of interacting molecule 1 from cluster 1 and interacting molecule 2 from cluster 2. (c) Dot plot visualization of markers expression from selected relevant interactions data across clusters. Shown are the expression levels of receptors and ligands for BMP, FGF, IGF, WNT and TGFβ pathways. The size of the circles indicates the percentage of expression. The grayscale bar indicates the mean values of the average expression levels.

FIG. 7: Transplantation of hESC-derived enriched thyroid follicles into NOD-SCID mice and in vivo functionality of transplanted human ESC-derived thyroid follicles. (a) Schematic representation of the hESC-derived thyroid follicles transplantation under the kidney capsule protocol in NOD-SCID untreated mice or previously thyroid radioactive iodine (RAI) ablated by intraperitoneal ¹³¹I injection. (b) Histological analysis shows the presence of active follicles with cuboidal to weakly columnar epithelium and inactive follicles with flat/scaly cell organization. (c) Gene expression levels of thyroid markers in transplanted tissue (n=4) compared to human thyroid tissue. An unpaired t-test was used for statistical analysis. *p<0.012. (d) Quantification of ¹²³I uptake in single-photon emission computed tomography (SPECT) images expressed as percentage of injected dose (% ID) in the intact (non-irradiated) (2.44 (1.72-4.91) % ID; n=5) or RAI-ablated thyroid region (0.01 (0.002-0.03) % ID; n=6), and grafted tissue (1.66 (0.96-2.14) % ID; n=4). (e) Comparison of plasma T4 levels among controls (3.63 (3.35-3.80) μg/dl; n=6), irradiated/non-transplanted (0.11 (0.06-0.23) μg/dl; n=6) and irradiated/transplanted mice (1.26 (0.86-2.49) μg/dl; n=4). (f) Plasma levels of T3 in controls (n=6), RAI-ablated (RAI; n=6) and RAI-ablated/grafted mice (graft; n=10). Measurements were performed five weeks after transplantation. Two-sided unpaired Mann-Whitney was used for statistical analysis (r and p values are presented in the graph; ns not significant; data are presented as median (IQR)). (g) Correlation of plasma TSH and T4 levels among grafted animals (n=10). Measurements were performed five weeks after transplantation. Two-sided unpaired Mann-Whitney was used for statistical analysis (r and p values are presented in the graph; ns not significant; data are presented as median (IQR)). (h) Comparison of hepatic Dio1 mRNA levels in controls (n=6), RAI-ablated (n=6) and RAI-ablated/transplanted mice (n=10). Measurements were performed five weeks after transplantation. Spearman Correlation test was used for statistical analysis (r and p values are presented in the graphs; ns not significant; data are presented as median (IQR)).

FIG. 8: (A) Schematic representation of a method for generating lung organoids from human ESCs according to an embodiment of the invention. (B-D) Bulk RNA sequencing data showing the expression profile of lung markers over time. Lung progenitors markers are more prominently expressed between day 23 and day 31 (B), while markers of airway proximal cells, such as basal, secretory, ciliated and neuroendocrine cells (C) are mainly expressed from day 31 and alveolar markers (AT1 and AT2) appearing from day 38 with overall higher levels at day 45 (D).

FIG. 9: Histological characterization of the lung structures generated in vitro at day 45 according to the protocol shown in FIG. 8A. Immunofluorescence analyses demonstrate epithelial structures expressing NKX2-1 and E-CADHERIN (A), basal cells expressing KRT5 and P63 (B), secretory cells expressing MUC5A5 inside of SOX2+ structures (C), ciliated cells with cilia expressing Acetylated tubulin (D), mixed structure containing MUC5A5 in the tube and HOPX+ cells on the top (E) and alveolar cells expressing HOPX (F). Electronic microscopy images evidence the presence of bronchi structures containing ciliated cells (G) and goblet cells producing mucus (H).

FIG. 10: Insulin removal experiments. (A) Schemes of the conditions used for the analysis. qPCR analyses (B-F) show that insulin treatment for only one week (day 9-16) strongly reduces the expression of markers for all lung cell types (B-F) while incubation with insulin for 2 weeks (day 9-23) seems to induce immature lung structures evidenced by TP63 high levels (B) while the other markers are significantly downregulated (C-F).

FIG. 11: scRNA-Seq characterization of human ESC-derived thyroid cells at day 58. (a) Heatmap showing normalized expression of selected marker genes with rows representing cell clusters, while columns represent genes. The intensity of the greyscale in each square indicates the mean expression within the cluster. (b) qRT-PCR analysis of PAX8, NIS, TSHR, TG, and TPO at thyroid organoids from day 58 of differentiation protocol compared to uninduced control (−Dox) (n=4).

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

Standard reference works setting forth the general principles of recombinant DNA technology include “Molecular Cloning: A Laboratory Manual, 4th Edition” (Green and Sambrook, 2012, Cold Spring Harbor Laboratory Press); Current Protocols in Molecular Biology, ed. Ausubel et al., Greene Publishing and Wiley-Interscience, New York, 1992 (with periodic updates) (“Ausubel et al. 1992”). Desai et al. (2015. Reproductive Biology and Endocrinology 13:9) have reviewed human embryonic stem cell culture. Efthymiou et al. (2014. Expert Opinion on Biological Therapy 14:1333-1344) have reviewed strategies to maintain stem cell pluripotency as well as cell lineage differentiation strategies in human embryonic stem cells and induced pluripotent stem cells.

The present invention provides methods for directing the differentiation of pluripotent stem cells (PSCs) into thyroid or lung organoids in vitro or ex vivo, thyroid and lung organoids obtainable by these methods and uses thereof. The present invention is in particular captured by any one or any combination of one or more of the below numbered aspects and embodiments with any other aspect, statement and/or embodiment:
1. A (in vitro) method for generating a thyroid organoid or a lung organoid from human pluripotent stem cells, said method comprising:
   a. generating an embryoid body (EB) from human pluripotent stem cells (hPSCs);
   b. culturing the embryoid body in a 3D matrix under conditions to induce differentiation into definitive endoderm;
   c. transiently overexpressing NKX2-1 and PAX8 in the definitive endoderm to induce differentiation of endodermal cells into thyroid and lung progenitor cells; and
   d. culturing the thyroid and lung progenitor cells in the 3D matrix in a differentiation medium for a period of time sufficient to generate a thyroid organoid or a lung organoid.
2. The method according to 1, wherein said human pluripotent stem cells are human embryonic stem cells.
3. The method according to 1 or 2, wherein the embryoid bodies are generated (step a) by a hanging drop culture.
4. The method according to any one of 1 to 3, wherein inducing differentiation of the embryoid body into definitive endoderm (step b) comprises contacting the embryoid body in the 3D matrix with Activin A.
5. The method according to 4, wherein the embryoid body is contacted with Activin A for about 1 to about 5 days, preferably for about 3 days.
6. The method according to any one of 1 to 5, wherein the 3D matrix is a 3D basement membrane matrix, preferably wherein the 3D matrix comprises Matrigel.
7. The method according to any one of 1 to 6, wherein the definitive endoderm comprises endodermal cells expressing SRY-Box Transcription Factor 17 (SOX17) and Forkhead Box A2 (FOXA2).
8. The method according to any one of 1 to 7, wherein the human pluripotent stem cells have been genetically modified with an inducible expression system encoding NKX2-1 and PAX8.
9. The method according to 8, wherein transient NKX2-1 and PAX8 overexpression in the endodermal cells (step c) comprises culturing the definitive endoderm in the 3D matrix in a basal differentiation medium supplemented with an agent that activates the inducible expression system.
10. The method according to any one 1 to 9, wherein NKX2-1 and PAX8 are overexpressed for between 4 and 10 days, preferably for 4 days.
11. The method according to any one of 1 to 10, wherein the thyroid and lung progenitor cells express NKX2-1 and PAX8.
12. The method according to any one of 1 to 11 for generating a thyroid organoid, wherein culturing the thyroid and lung progenitor cells (step d) comprises culturing in a first differentiation medium comprising a basal differentiation medium and a cyclic monophosphate (cAMP), and subsequent culturing in a second differentiation medium comprising the basal differentiation medium and a glucocorticoid and a thyroid-stimulating hormone.
13. The method according to 12, wherein a TGF-beta inhibitor is added to the second differentiation medium after about 7 days of culture in said second differentiation medium.
14. The method according to 12 or 13, wherein the glucocorticoid is dexamethasone.
15. The method according to any one of 12 to 14, wherein the thyroid-stimulating hormone is thyrotropin.
16. The method according to any one of 12 to 15, wherein the thyroid and lung progenitor cells are cultured in the first differentiation medium for about 14 days and in the second differentiation medium for at least about 28 days
17. The method according to any one of 12 to 16, wherein step d further comprises culturing the thyroid and lung progenitor cells in the basal differentiation medium for between about 5 and about 9 days, preferably about 7 days, before culturing them in the first differentiation medium.
18. The method according to any one of 1 to 11 for generating a lung organoid, wherein culturing the thyroid and lung progenitor cells (step d) comprises culturing in a differentiation medium comprising a basal differentiation medium, a cyclic monophosphate (cAMP), and insulin or insulin-like growth factor 1 (IGF-1).
19. The method according to 18, wherein a TGF-beta inhibitor is added to the differentiation medium after about 28 days of culture in the differentiation medium.
20. The method according to 18 or 19, wherein the thyroid and lung progenitor cells are cultured in the differentiation medium for at least about 36 days.
21. The method according to any one of 18 to 20, wherein step d consists of culturing the thyroid and lung progenitor cells in the 3D matrix in a differentiation medium for a period of time sufficient to generate a thyroid organoid or a lung organoid. In particular embodiments, a thyroid organoid is generated from the hPSCs in less than 60 days, such as in 58 or 59 days. In particular embodiments, a lung organoid is generated from the hPSCs in less than 50 days, such as in 45, 46, 47, 48 or 49 days.
22. A thyroid organoid obtainable by the method according to any one of 1 to 17. In preferred embodiments, the thyroid organoid is an in vitro or ex vivo generated thyroid organoid, and/or is a hPSC-derived, in particular a hESC-derived, thyroid organoid.
23. The thyroid organoid according to 22, in which at least 10%, preferably at least 15%, more preferably at least 20% or 25%, of the cells express one or more thyroid markers selected from the group consisting of NKX2-1, PAX8, Forkhead Box E1 (FOXE1), Thyroid Stimulating Hormone Receptor (TSHR), thyroglobulin (TG) and thyroid peroxidase (TPO).
24. The thyroid organoid according to 22 or 23, which exhibits a spheroidal structure comprising a single layer of cells and a lumen.
25. The thyroid organoid according to any one of 22 to 24, which is capable to synthesize a thyroid hormone in vitro and in vivo.
26. A thyroid organoid according to any one of 22 to 25 for use in medicine.
27. A thyroid organoid according to any one of 22 to 25 for use in the prevention or treatment of a thyroid disease or disorder a subject.
28. The thyroid organoid for use according to 27 wherein said thyroid disease or disorder is hypothyroidism.
29. Use of a thyroid organoid according to any one of 22 to 25 as a model system for studying thyroid development.
30. Use of a thyroid organoid according to any one of 22 to 25 as a model system for studying a thyroid disease or disorder.
31. Use of a thyroid organoid according to any one of 22 to 25 as a platform for screening for drugs for the treatment of a thyroid disease or disorder.
32. Use of thyroid organoid according to any one of 22 to 25 as a platform for testing the effect of a test compound on thyroid tissue, preferably wherein said effect is selected from the group comprising a toxic effect, an effect on thyroid development and a pharmacological effect.
33. A method for studying the effect of a test compound on thyroid tissue, said method comprising:
   contacting the test compound with a thyroid organoid according to any one of 22 to 25, and
   determining the effect of the test compound on the thyroid organoid,
   preferably wherein said effect is selected from the group comprising a toxic effect, an effect on thyroid development and a pharmacological effect.
34. A lung organoid obtainable by the method according to any one of 1 to 11, or 18 to 21. In preferred embodiments, the lung organoid is an in vitro or ex vivo generated lung organoid, and/or is a hPSC-derived, in particular a hESC-derived, lung organoid.
35. The lung organoid according to 34, which comprises basal cells, ciliated cells, neuroendocrine cells, secretory cells, alveolar type 1 cells and alveolar type 2 cells.
36. The lung organoid according to 34 or 35, wherein the basal cells express one or more genes selected from the group consisting of keratin 5 (KRT5) and tumor protein (TP63), preferably TP63.
37. The lung organoid according to any one of 34 to 36, wherein the ciliated cells express one or more genes selected from the group consisting of Forkhead Box J1 (FOXJ1), Tubulin Beta 6 Class V (TUBB6) and Neuronal Calcium Sensor 1 (NCS1), preferably FOXJ1.
38. The lung organoid according to any one of 34 to 37, wherein the neuroendocrine cells express one or more genes selected from the group consisting of Ubiquitin C-Terminal Hydrolase L1 (UCHL1), Delta Like Canonical Notch Ligand 1 (DLL1) and Achaete-Scute Family BHLH Transcription Factor 1 (ASCL1).
39. The lung organoid according to any one of 34 to 38, wherein the secretory cells express one or more genes selected from the group consisting of SRY-box transcription factor 2 (SOX2), Secretoglobin Family 3A Member 2 (SCGB3a2), mucin 1 (MUC1), mucin 5AC (MUC5AC), mucing 5B (MUC5B), cystic fibrosis transmembrane conductance regulator (CTFR) and Anterior Gradient 2 (AGR2), preferably SCGB3a2.
40. The lung organoid according to any one of 34 to 39, wherein the alveolar type 1 cells express one or more genes selected from the group consisting of A-Kinase Anchoring Protein 5 (AKAP5), Chloride Intracellular Channel 5 (CLIC5), aquaporin 5 (AQP5), homeodomain-only protein homeobox (HOPX), podoplanin (PDPN), Epithelial Membrane Protein 2 (EMP2), Crystallin Alpha B (CRYAB) and advanced glycosylation end-product specific receptor (AGER), preferably AGER.
41. The lung organoid according to any one of 34 to 40, wherein the alveolar type 2 cells express one or more genes selected from the group consisting of Surfactant Protein B (SFTPB), C-X-C Motif Chemokine Ligand 5 (CXCL5), secretory leukocyte protease inhibitor (SLP1), Surfactant Protein A1 (SFTPA1), proliferator-activated receptor γ coactivator (PGC), claudin 18 (CLDN18), ATP Binding Cassette Subfamily A Member 3 (ABCA3), Lysosomal Associated Membrane Protein 3 (LAMP3), Solute Carrier Family 34 Member 2 (SLC34A2), napsin A aspartic peptidase (NAPSA) and Lysophosphatidylcholine Acyltransferase 1 (LPCAT1), preferably LAMP3.
42. Use of a lung organoid according to any one of 34 to 41 as a model system for studying lung development.
43. Use of a lung organoid according to any one of 34 to 41 as a model system for studying a lung disease or disorder.
44. Use of a lung organoid according to any one of 34 to 41 as a platform for screening for drugs for the treatment of a lung disease or disorder.
45. Use of a lung organoid according to any one of 34 to 41 as a platform for testing the effect of a test compound on lung tissue, preferably wherein said effect is selected from the group comprising a toxic effect, an effect on lung development and a pharmacological effect.
46. A method for studying the effect of a test compound on lung tissue, said method comprising:
   contacting the test compound with a lung organoid according to any one of 34 to 41, and
   determining the effect of the test compound on the lung organoid,
   preferably wherein said effect is selected from the group comprising a toxic effect, an effect on lung development and a pharmacological effect.
47. A lung organoid according to any one of 34 to 41 for use in medicine.
48. A lung organoid according to any one of 34 to 41 for use in the prevention or treatment of a lung disease or disorder a subject.

In embodiments, (human) PSCs, such as (human) ESCs, undergo directed differentiation in a step-wise manner, first into embryoid bodies, then into definitive endoderm, then into thyroid and lung precursor cells and then into thyroid or lung organoids.

In developmental biology, cellular differentiation is the process by which a less specialized cell becomes a more specialized cell type. As used herein, the term “directed differentiation” describes a process through which a less specialized cell becomes a particular specialized target cell type. In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a thyroid and lung progenitor cell), which in turn can differentiate into other types of precursor cells further down the pathway (such as a thyroid precursor or a lung precursor), and then to an end-stage differentiated cell (such as a thyroid follicular epithelial cell) which plays a characteristic role in a certain tissue type, and may or may not retain the capacity to proliferate further.

As used herein, the term “organoid” is used to mean a 3-dimensional growth of cells in culture that retains characteristics of the corresponding tissue and organ in vivo, e.g. prolonged tissue expansion with proliferation, multilineage differentiation, and/or recapitulation of cellular and tissue ultrastructure, etc.

The term “pluripotent stem cells” denote cells having a pluripotent differentiation and self-renewal ability. Pluripotent stem cells have the potential to differentiate into any of the three germ layers: endoderm (interior stomach lining, gastrointestinal tract, the lungs), mesoderm (muscle, bone, blood, urogenital), and ectoderm (epidermal tissues and nervous system). As such, pluripotent stem cells can give rise to any fetal or adult cell type. Non-limiting examples of pluripotent stem cells that can be used in the methods described herein include (human) embryonic stem cells (ES cells), epiblast stem cells (EpiS cells), embryonal carcinoma cells (EC cells), embryonic germ cells (EG cells), multipotent germline stem cells (mGS cells), and MUSE cells (see Kuroda Y. et al., Proc. Natl. Acad. Sci. U.S.A., 2010, Volume 107, Issue 19, pages 8639 to 8643). Moreover, “pluripotent stem cells” as used herein also include cells artificially induced to have pluripotency from somatic cells collected from a living body, such as induced pluripotent stem cells (iPS cells and the like).

In preferred embodiments, the pluripotent stem cells are (human) embryonic stem cells.

The term “embryonic stem cells” refers to cells that are pluripotent and are derived from tissues formed before the end of pregnancy after fertilization, including tissues taken at any time during pregnancy (usually but not necessarily not before about 10-12 weeks of pregnancy) such as fetal tissue, pre-embryonic tissue (for example, blastocyst) or embryonic tissue. Embryonic stem cells can be obtained directly from suitable tissues (including but not limited to human tissues) or from established embryonic stem cell lines or through the single blastomere biopsy method without (human) embryo destruction as e.g. reported by Chung et al. (2008. Cell Stem Cell 2(2): 113-117. Methods for deriving embryonic stem cells from blastocytes are well known in the art. For example, three cell lines (H1, H13, and H14) have a normal XY karyotype, and two cell lines (H7 and H9) have a normal XX karyotype. Additional embryonic stem cells that can be used in the methods described herein include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Indeed, embryonic stem cells that can be used in the methods described herein include but are not limited to SA01 (SA001); SA02 (SA002); ES01 (HES-1); ES02 (HES-2); ES03 (HES-3); ESO4 (HES-4); ES05 (HES-5); ES06 (HES-6); BG01 (BGN-01); BG02 (BGN-02); BG03 (BGN-03); TE03 (13); TE04 (14); TE06 (16); UC01 (HSF1); UC06 (HSF6); WA01 (H1); WA07 (H7); WA09 (H9); WA13 (H13); WA14 (H14). More details on embryonic stem cells can be found in, for example, Thomson et al., 1998, Science 282 (5391):1145-1147; Andrews et al., 2005, Biochem Soc Trans 33:1526-1530; Martin 1980, Science 209 (4458):768-776; Evans and Kaufman, 1981, Nature 292(5819): 154-156; Klimanskaya et al., 2005, Lancet 365 (9471): 1636-1641).

The origin of the pluripotent stem cells is not particularly limited, and includes humans and non-human animals. In particular embodiments, the pluripotent stem cells are from a mammal (i.e. mammalian pluripotent stem cells), such as any mammal (e.g., murine, bovine, ovine, porcine, canine, feline, equine, primate (e.g. human)), preferably a human.

In preferred embodiments, the pluripotent stem cells are human pluripotent stem cells, in particular human embryonic stem cells.

The pluripotent stem cells as used herein may be cells that have been genetically modified. The genetically modified cells may be cells exogenously introduced with a gene encoding a protein to be (over) expressed, may be cells in which the function of a specific gene is suppressed by genome editing, knockout method, RNA method, antisense method, or the like, and/or may be cells in which the function of a gene is randomly suppressed or activated, or the like.

In particular embodiments, the (human) pluripotent stem cells are genetically modified to inducibly overexpress the transcription factors NKX2-1 and PAX 8.

In further particular embodiments, the (human) pluripotent stem cells are genetically modified with a nucleic acid construct comprising the coding sequence for NKX2-1 and/or, preferably and, the coding sequence for PAX 8 operably linked to an inducible gene expression system such as, e.g., a Tet-On system. The transcription factors NKX2-1 and PAX 8 may include those encoded in the human genome as well as homologs and orthologs from other species. Efficiency can be improved through the use of linkers to co-express the transcription factors with the use of internal ribosome entry site (IRES) as known to the skilled person.

Tools for inducible gene expression are well known in the art and include, for example but without limitation, Tet-On systems (e.g. an doxycycline-inducible TRE-rtTA system) and Tet-Off systems. “Tet-On” and “Tet-off” systems as used herein refer to tetracycline-responsive promoter element (TRE)-regulated expression systems. The term “tetracycline-responsive promoter element” (abbreviated ““TRE”) refers to a promoter sequence comprising one, or more (such as, e.g., two, three, four, five, six, seven or more, for instance two or seven) tetracycline operator (tetO) sequences. A tetO is a polynucleotide segment that is capable of binding to a tetO-binding protein such as, e.g., a tetracycline repressor (tetR), tetracycline-controlled transcriptional activator (tTA) or a reverse tTA (rtTA) protein. Expression of a gene/genes of interest becomes inducible by the co-expression of proteins binding to (tetracycline operator (tetO) sequences within) the TRE, such as, e.g., a tetracycline repressor (tetR), a tetracycline-controlled transcriptional activator (tTA) or a reverse tTA (rtTA) and the presence or absence of tetracycline or a tetracycline derivative such as, e.g., doxycycline.

### Formation of Embryoid Bodies

In the methods described herein, the (human) pluripotent stem cells are cultured under conditions to form embryoid bodies. As used herein, an “embryoid body” or “EB” refers to a 3-dimensional aggregate of cells that mimics some structure of a developing embryo and can differentiate into cells of all three germ layers.

The method for culturing pluripotent stem cells is not particularly limited as long as embryoid bodies can be formed, and may include, for example, suspension culture, hanging drop culture or the use of non-adhesive microwell structures as known to the skilled person. Those skilled in the art can appropriately select an embryoid body culture method. In the suspension method pluripotent stem cells may be kept in suspension on a cell substrate (e.g. through addition of soluble factors in the culture medium that prevent cell attachment and/or promote cell-cell interactions, and/or using a non-adherent cell substrate) and allowed to self-aggregate into EBs. The resulting EBs may be heterogenous. In the hanging drop method a suspension of pluripotent stem cells is cultured in a drop on a surface turned upside down and EBs form through aggregation at the bottom of the drops. To obtain more homogenous EBs, and in a more reproducible way, non-adhesive microwell structures may be used. In this technique, small wells (e.g. in the submillimeter range) are produced and cells are seeded inside. The cells then aggregate and grow until they are limited by the size of the microwells. A non-limiting example is the agarose microwell array disclosed in Pettinato et al. (2014. Formation of Well-defined Embryoid Bodies form Dissociated Human Induced Pluripotent Stem Cells using Microfabricated Cell-repellent Microwel Arrays. Scientific Reports 4:7402). In embodiments, embryoid bodies are formed from the (human) pluripotent stem cells by a hanging drop culture.

The medium used in such culture can be prepared based on a known basal medium for culturing pluripotent stem cells, in particular human pluripotent stem cells, which may be a nutrient-rich buffered solution capable of sustaining cell growth. Non-limiting examples of a basal medium for culturing human pluripotent stem cells include a commercially available medium such as StemFlex™, DMEM medium, KSOM medium, Eagle's MEM medium, Glasgow MEM medium, αMEM medium, Ham medium, RPMI 1640 medium, Fisher's medium, BME medium, BGJb medium, CMRL 1066 medium, MEM Zinc optional improvement medium, IMDM medium, Medium 199 medium, and any mixed medium thereof. The medium for forming an embryoid body may be a serum-containing medium or a serum-free medium, or may contain a serum replacement. In addition, it is possible to contain one or more of amino acids (such as L-glutamine and non-essential amino acids), reducing agents (such as β-mercaptoethanol), antibiotics (such as penicillin and streptomycin), hormones, growth factors, organic acids, fatty acids or lipids, saccharides, nucleosides, vitamins, cytokines, antioxidants, buffer agents, inorganic salts, and the like.

The basal medium may be supplemented with a compound that improves cell survival of the pluripotent stem cells such as, e.g. a Rho-associated coiled-coil kinase (Rock) inhibitor.

The basal medium may be supplemented with a compound that promotes cell aggregation and cell-cell adhesion such as, e.g., polyvinyl alcohol (PVA).

In embodiments of the methods described herein, a basal culture medium, preferably StemFlex™, is supplemented with a Rock inhibitor, preferably at a concentration of between 2 μM and 20 μM, more preferably at a concentration of about 10 μM, and/or, preferably and, polyvinyl alcohol (PVA), preferably at a concentration between 1 mg/ml and 10 mg/ml, more preferably at a concentration of about 4 mg/ml, for the formation of embryoid bodies.

Other conditions for forming embryoid bodies can be appropriately selected and adjusted by those skilled in the art according to known culture conditions. For example, the culture temperature is not particularly limited, but is usually about 30 to 40° C., and preferably about 37° C. The concentration of CO₂ is usually about 1 to 10%, and preferably about 2 to 5%.

The number of pluripotent stem cells provided for forming an embryoid body is not particularly limited as long as the embryoid body can be formed, and can be appropriately adjusted by those skilled in the art according to the type and origin of the pluripotent stem cells, as well as the culturing method, and may range from 50 to 50000. For hanging drop culture of pluripotent stem cells such as embryonic stem cells, from 100 to 10000, preferably from 500 to 5000, more preferably about 1000 pluripotent stem cells may be cultured to form an embryoid body.

The culture period for forming embryoid bodies is not particularly limited, and can be appropriately adjusted by those skilled in the art according to the type and origin of the pluripotent stem cells to be used, as wells as the selected culture method and may range between 1 to 14 days, preferably 1 to 7 days, more preferably 2 to 5 days such as 2, 3, 4 or 5 days, even more preferably 2 days.

The formation of embryonic bodies can be monitored according to a conventional method by morphological evaluation (for example, histological staining).

### Differentiation Into Definitive Endoderm

In the methods described herein, embryoid bodies are cultured under conditions for the embryoid bodies to differentiate into definitive endoderm.

As used herein, “definitive endoderm” or “DE” refers to one of the three germ layers arising after gastrulation that give rise to organs of the gastrointestinal and respiratory tract including e.g. the intestinal tract, liver, pancreas, stomach, thyroid, lung.

Differentiation into definitive endoderm may be confirmed by determining the expression of one or more differentiation-specific markers selected from the group consisting of SOX17 and FOXA2, preferably of SOX17 and FOXA2. The expression of the differentiation-specific marker can be determined at mRNA level or at protein level, e.g. by an immunological measurement (e.g. immunofluorescence (IF)) or an RNA expression analysis (for example, RT-(q)PCR and cDNA microarray).

Methods for inducing differentiation into definitive endoderm are known to the skilled person, and include, without limitation, the addition of one or more suitable growth factors (e.g. growth factors from the TGF-β superfamily such as Nodal/Activin and/or BMP, more particularly Nodal, Activin A, Activin B, BMP4, Wnt3a) and/or small molecules (e.g. sodium butyrate, IDE 1) to a basal differentiation medium, or using commercially available kits (e.g. Gibco™ PSC Definitive Endoderm Induction Kit). Any such method for producing definitive endoderm from pluripotent cells is applicable to the methods described herein. For example, Activin A treatment is a well-known method for inducing definitive endoderm (see e.g. Bogacheva et al. 2018. J Cell Physiol. 233:3578-3589). Also combinations of Activin A and a glycogen synthase kinase (GSK)-3 inhibitor (e.g. CHIR99021 and 6-bromoindirubin-3′-oxime (BIO)) or bone morphogenetic protein 4 (BMP4) can induce definitive endoderm (Teo et al. 2014. Stem Cell Reports 3:5-14), or combinations of growth factors (Activin A and Wnt-3a) and small molecules (sodium butyrate and IDE 1) (Bogacheva et al. 2018).

In embodiments of the methods described herein, definitive endoderm may be induced by contacting the generated embryoid bodies with Activin A. In particular, the embryoid bodies may be cultured in a basal differentiation medium supplemented with Activin A at a concentration of between 50 ng/ml and 100 ng/ml, preferably about 50 ng/ml.

As a basal medium for differentiation, any of media usable for animal cell culture, in particular human cell culture, such as DMEM medium, BME medium, αMEM medium, serum-free DMEM/F12 medium, BGJb medium, CMRL 1066 medium, Glasgow MEM medium, Improved MEM Zinc Option medium, IMDM medium, Medium 199 medium, Eagle MEM medium, Ham's medium, RPMI 1640 medium, Fischer's medium, McCoy's medium, and Williams E medium, may be used. The basal differentiation medium may be a serum-containing medium or a serum-free medium, or may contain a serum replacement. Various nutrient sources necessary for the maintenance and growth of cells may be properly added to the medium. For example, as the nutrient sources, the medium may contain one or more of: carbon sources, such as glycerol, glucose, fructose, sucrose, lactose, sodium pyruvate, honey, starch, and dextrin; carbohydrates, such as fatty acid, oil and fat, lecithin, and alcohol; nitrogen sources, such as ammonium sulfate, ammonium nitrate, ammonium chloride, urea, and sodium nitrate; inorganic salts, such as common salt, potassium salt, phosphate, magnesium salt, calcium salt, iron salt, and manganese salt; monopotassium phosphate, dipotassium phosphate, magnesium sulfate, sodium chloride, ferrous sulfate, sodium molybdate, sodium tungstate, and manganese sulfate; vitamins, such as vitamin C; and amino acids, such as L-glutamine and non-essential amino acids. In addition, it is possible to contain one or more of reducing agents (such as β-mercaptoethanol), antibiotics (such as penicillin and streptomycin), antioxidants, buffer agents, and the like.

A suitable basal differentiation medium for use with the methods described herein may be a DMEM/F12 medium supplemented with serum, such as fetal bovine serum (FBS); amino acids, such as L-glutamine and non-essential amino acids; antibiotics, such as penicillin and streptomycin; a reducing agent, such as 2-mercaptoethanol; vitamins, such as vitamin; and a carbon source, such as sodium pyruvate; e.g. DMEM/F12+Glutamax (Gibco) with 20% (v/v) FBS (Gibco), 0.1 mM MEM-Non-Essential Amino Acids (Gibco), 1 mM sodium pyruvate (Gibco), 0.1 mM 2-mercaptoethanol (Sigma), 100 U/ml Penicillin-Streptomycin (Gibco), and 50 μg/ml L-ascorbic acid (Sigma).

The treatment with Activin may last for about 2 days to about 5 days, preferably for about 2 days to about 4 days, more preferably for about 3 days.

Preferably, the embryoid bodies are cultured in a three-dimensional (3D) matrix, i.e. the embryoid bodies are embedded in a 3D matrix, to induce definitive endoderm. The 3D matrix may include one or more extracellular matrix (ECM) proteins. The 3D matrix may include, without limitation, matrigel, fibronectin, collagen (e.g., collagen I, collagen IV, etc.), collagen derivatives, gelatin, laminin, heparan sulfate proteoglycans, entactin/nidogen, cellulose, cellulose derivatives, cellulose polymers, proteoglycans, heparin sulfate, chondroitin sulfate, keratin sulfate, hyaluronic acid, elastin, fibrin, chitosan, alginate, vinculin, agar, agarose, and combinations thereof. The 3D matrix may comprise one or more polymers including, for example, polyethylene-imine and dextran sulfate, poly(vinylsiloxane)ecopolymerepoly-ethyleneimine, phosphorylcholine, poly(ethylene glycol), poly(lactic-glycolic acid), poly (lactic acid), polyhydroxy valerte and copolymers, polyhydroxybutyrate and copolymers, polydiaxanone, poly anhydrides, polypeptides, poly(orthoesters), polyesters, and combinations thereof. In preferred embodiments, the 3D matrix is a 3D basement membrane matrix, more preferably the 3D matrix comprises Matrigel. Matrigel may comprise laminin, collagen IV, heparan sulfate proteoglycans, entactin/nidogen, TGF-beta, epidermal growth factor, insulin-like growth factor, fibroblast growth factor, and tissue plasminogen activator, additional proteins, or combinations thereof.

### Induced NKX2-1 and PAX 8 (Over) Expression

In order to commit the definitive endoderm to thyroid and lung lineages, NKX2-1 and PAX 8 are transiently (over) expressed in the methods described herein. “Transient” gene (over) expression or “transient” protein (over) expression as used herein refers to the temporary expression of a gene or protein of interest that is expressed for a short time after (over) expression of the gene or protein has been induced. By inducibly (over) expressing a gene or protein of interest is meant herein that (over) expression of the gene or protein of interest is at least in part influenced by at least one external stimulus. Non-limiting examples of such an external stimulus are substances, (e.g. growth factors or oligonucleotides such as antisense oligonucleotides), including activators and inhibitors, that interfere with signalling pathways which in the end lead to activation of NKX2-1 and PAX 8 expression; inducers of inducible expression systems as known in the art; the introduction of an expression cassette encoding the protein of interest or the introduction of an mRNA molecule that can be translated in the protein of interest.

In embodiments, bone morphogenetic protein (BMP) signalling and fibroblast growth factor (FGF) signalling, and optionally Wnt signalling, is activated in the definitive endoderm cells to transiently overexpress NKX2-1 and PAX8, e.g. by contacting the definitive endoderm cells with fibroblast growth factor 2 (FGF2) and bone morphogenetic protein 4 (BMP4) and optionally Wnt3a.

In other embodiments, Wnt signalling and bone morphogenetic protein (BMP) signalling are activated in the definitive endoderm cells to transiently overexpress NKX2-1 and PAX8, e.g. by contacting the definitive endoderm cells with CHIR99021 (an inhibitor of glycogen synthase kinase 3), retinoic acid (RA) and bone morphogenetic protein 4 (BMP4).

In embodiments, transcriptional co-activator with PDZ-binding motif (TAZ), which is a transcriptional co-activator that regulates several transcription factors, including PAX8 and NKX2-1 (Ma et al. 2017 Thyroid 27:292-299), may be activated, e.g. by contacting the definitive endoderm cells with ethacridine.

Technologies to transiently modify gene and/or protein expression in a cell of interest are known to the skilled person. For example, an expression cassette encoding the protein of interest may be introduced into the definitive endoderm cells by means of e.g. viral vectors. For transient overexpression of a protein, a non-integrating RNA-based approach can also be used. In particular, in vitro transcribed optimised mRNA molecules, such as mRNA molecules with modified 5′-and 3′UTRs, that can be translated into NKX2-1 and PAX 8 can be used to transfect the definitive endoderm cells, e.g. by electroporation, lipofection or cationic nanoparticles.

As described elsewhere herein, the pluripotent stem cells used in the methods described herein may be genetically modified to inducibly overexpress the transcription factors NKX2-1 and PAX 8. In particular embodiments, the pluripotent stem cells are genetically modified with a nucleic acid construct comprising an open reading frame encoding NKX2-1 and/or, preferably and, an open reading frame encoding PAX 8 operably linked to an inducible gene expression system. Accordingly, in embodiments, (over) expression of NKX2-1 and PAX 8 is induced by contacting the definitive endoderm with an inducing agent that is capable to drive expression of the open reading frames encoding NKX2-1 and PAX 8 from the construct. The inducing agent may be added to a basal differentiation medium as described elsewhere herein and the definitive endoderm may be cultured in the basal differentiation medium supplemented with the inducer (e.g. doxycycline). In particular embodiments wherein the pluripotent stem cells are genetically modified with a nucleic acid construct comprising an open reading frame encoding NKX2-1 and an open reading frame encoding PAX 8, which open reading frames are operably linked to a Tet-On system (e.g. an doxycycline-inducible TRE-rtTA system), inducing NKX2-1 and PAX 8 expression in the definitive endoderm may comprise culturing the definitive endoderm in a basal differentiation medium as described elsewhere herein supplemented with doxycycline, preferably at a concentration of between 0.1 mg/ml and 10 mg/ml, preferably between 0.5 mg/ml and 5 mg/ml, more preferably about 1 mg/ml.

Preferably, the definitive endoderm is cultured in a three-dimensional (3D) matrix as described elsewhere herein.

In embodiments, NKX2-1 and PAX 8 are transiently (over) expressed during maximal 10 days, preferably during between 4 to 10 days, more preferably during between 4 to 8 days, during between 4 to 7 days, during between 4 to 6 days or during between 4 to 5 days, even more preferably during 4 days. In particular embodiments, the definitive endoderm is cultured in a basal differentiation medium supplemented with an inducing agent, in particular doxycycline, for maximal 10 days, preferably for between 4 to 10 days, more preferably for between 4 to 8 days, for between 4 to 7 days, for between 4 to 6 days or for between 4 to 5 days, even more preferably for 4 days.

Induced (over) expression of NKX2-1 and PAX 8 committed endodermal cells to differentiate into thyroid and lung progenitor cells. The thyroid and lung progenitor cells express markers NKX2-1 and PAX 8. Expression of the markers can be determined by an immunological measurement (e.g. IF) or an RNA expression analysis (for example, RT-(q)PCR and cDNA microarray).

### Thyroid Differentiation

For further differentiation into thyroid organoids, the cell culture or the cell population enriched in thyroid and lung progenitor cells, is further cultured in a 3D matrix as described elsewhere herein in a suitable differentiation medium for a period of time sufficient to obtain thyroid organoids.

As shown in the experimental section, culturing the cells in the presence of a cyclic monophosphate, a glucocorticoid (e.g. dexamethasone), a thyroid-stimulating hormone (e.g. rhTSH) and a TGF-β inhibitor lead the cells to form fully functional thyroid follicles. In embodiments, the cell culture or the cell population enriched in thyroid and lung progenitor cells is cultured in the presence of a cyclic monophosphate and subsequently cultured in the presence of a glucocorticoid and a thyroid hormone. To achieve full maturation of the thyroid follicles, a TGF-β inhibitor may be added to the culture.

The culture in the presence of a cyclic monophosphate, a glucocorticoid, a thyroid-stimulating hormone and/or a TGF-β inhibitor may be achieved by adding a cyclic monophosphate (e.g. 8-br-CAMP, 2-br-cAMP, dibutyril-cAMP (sodium salt)), a glucocorticoid (e.g. dexamethasone, beclomethasone, betamethasone, budesonide, cortisone, hydrocortisone, methylprednisolone, prednisolone, prednisone and triamcinolone), a thyroid-stimulating hormone (e.g. rhTSH, bovine TSH, human TSH) and/or a TGF-β inhibitor (e.g. SB431542; TGF-β RI Kinase Inhibitor VIII (CAS 356559-20-1); SB-505124 hydrochloride hydrate; LY2109761; galunisertib; A 83-01 (sodium salt); TGFβRI-IN-3; RepSox; SB 525334; vactosertib (hydrochloride); SB-5025124; LY-364947; GW788388; LSKL, Inhibitor of Thrombospondin (TSP-1); LY3200882; ITD-1; PF-06952229; SD-208; R-268712; SM 16; AZ12601011; A 77-01; BIBF0775; IN-1130; pm26TGF-β1 peptide TFA; TGFβRI-IN-1; EW-7195) to a basal differentiation medium as described elsewhere herein at a concentration that can induce differentiation of the cell population into thyroid organoids. In particular embodiments, the cell culture or cell population is treated with a cyclic monophosphate at a concentration of at least 10 μM, at least 50 μM or at least 100 μM such as at a concentration between 100 μM and 1000 μM or between 100 μM and 500 μM, e.g. at a concentration of about 300 μM. In particular embodiments, the cell culture or cell population is treated with a thyroid-stimulating hormone at a concentration of at least 0.1 mU/ml, at least 0.2 mU/ml, at least 0.5 mU/ml or at least 1.0 mU/ml such as at a concentration between 0.5 mU/ml and 10 mU/ml or between 0.5 mU/ml and 5 mU/ml, e.g. at a concentration of about 1 mU/ml. In particular embodiments, the cell culture or cell population is treated with a glucocorticoid at a concentration of at least 5 nM, at least 10 nM, at least 20 nM or at least 50 nM such as at a concentration between 20 nM and 500 nM, between 20 nM and 200 nM or between 20 nM and 100 nM, e.g at a concentration of about 50 nM. In particular embodiments, the cell culture or cell population is treated with a TGF-β inhibitor at a concentration of at least 1 μM, at least 2 μM or at least 5 μM such as at a concentration between 1 μM and 100 μM, between 1 μM and 50 μM or between 1 μM and 20 μM, e.g. at a concentration of about 10 μM.

In particular embodiments, the cyclic monophosphate is 2-br-cAMP or 8-br-cAMP, preferably 8-br-cAMP. In particular embodiments, the glucocorticoid is dexamethasone. In particular embodiments, the TGF-β inhibitor is SB431542.

In further embodiments, the cell culture or the cell population enriched in thyroid and lung progenitor cells is cultured for between 7 and 21 days, preferably for between 10 and 18 days, more preferably for 14 days, in the presence of cyclic monophosphate, and subsequently for at least 21 days, preferably at least 28 days in the presence of a glucocorticoid and a thyroid-stimulating hormone. To achieve full maturation of the thyroid organoids, a TGF-beta inhibitor may be added to the culture after at least 5 days, 6 days or 7 days culturing in the presence of the glucocorticoid and the thyroid-stimulating hormone. Accordingly, in certain embodiments, the cell culture or the cell population enriched in thyroid and lung progenitor cells is subsequently cultured for between 7 and 21 days, preferably for between 10 and 18 days, more preferably for 14 days, in the presence of cyclic monophosphate, for between 5 and 10 days, preferably for about 7 days in the presence of a glucocorticoid and a thyroid-stimulating hormone, and for at least 14 days, preferably for at last 21 days in the presence a glucocorticoid, a thyroid-stimulating hormone and a TGF-β inhibitor. In particular embodiments, the cell culture or the cell population enriched in thyroid and lung progenitor cells is subsequently cultured for about 14 days, in the presence of cyclic monophosphate, for about 7 days in the presence of a glucocorticoid and a thyroid-stimulating hormone, and for at least 21 days in the presence a glucocorticoid, a thyroid-stimulating hormone and a TGF-β inhibitor.

The cell culture or the cell population enriched in thyroid and lung progenitor cells may be expanded prior to inducing differentiation into thyroid organoids, e.g. by culturing the cells in a 3D matrix as described elsewhere herein in a basal differentiation medium (without a differentiation inducer). Accordingly, in certain embodiments, the cell culture or the cell population enriched in thyroid and lung progenitor cells is cultured in a basal differentiation medium prior to culturing in the presence of a cyclic monophosphate. In further embodiments, the cell culture or the cell population enriched in thyroid and lung progenitor cells is cultured in the basal differentiation medium for between 1 and 14 days, preferably for between 1 and 10 days such as between 2 and 10 days, between 3 and 10 days, between 4 and 10 days or between 5 and 10 days, more preferably for about 7 days, prior to culturing in the presence of the cyclic monophosphate.

### Lung Differentiation

For differentiation into lung organoids, the cell culture or the cell population enriched in thyroid and lung progenitor cells were contacted with a cyclic monophosphate (cAMP) (e.g. 8-Br-cAMP), and insulin or insulin-like growth factor 1 (IGF1). To achieve full maturation of the lung organoids, a TGF-beta inhibitor may be added to the culture. Accordingly, in embodiments, the cell culture or the cell population enriched in thyroid and lung progenitor cells is cultured in the presence of a cyclic monophosphate, and insulin or IGF1. In further embodiments, a TGF-β inhibitor may be added to the culture.

The cell culture or the cell population enriched in thyroid and lung progenitor cells are preferably cultured in a 3D matrix as described elsewhere herein.

The cyclic monophosphate, the insulin or the IGF-1, and/or the TGF-β inhibitor may be added to a basal differentiation medium as described elsewhere herein at a concentration that can induce differentiation of the cell population into lung organoids. In particular embodiments, the cell culture or the cell population enriched in thyroid and lung progenitor cells are treated with insulin or IGF-1 at a concentration of 100 ng/ml or higher, 200 ng/ml or higher, 500 ng/ml or higher or 1 μg/ml or higher such as at a concentration between 100 ng/ml and 10 μg/ml or between 1 μg/ml and 10 μg/ml, e.g. at a concentration 5 μg/ml. In particular embodiments, the cell culture or the cell population enriched in thyroid and lung progenitor cells are treated with a cyclic monophosphate at a concentration of at least 10 μM, at least 50 μM or at least 100 μM such as at a concentration between 100 μM and 1000 μM or between 100 μM and 500 μM, e.g. at a concentration of about 300 μM. The concentration of each of the insulin or the IGF-1, and the cyclic monophosphate independently may be maintained at a constant level throughout the treatment, or may be varied during the course of the treatment. The concentration of each of the cyclic monophosphate, and the insulin or the IGF-1, may be varied independently. In particular embodiments, the cell culture or cell population is treated with a TGF-β inhibitor at a concentration of at least 1 μM, at least 2 μM or at least 5 μM such as at a concentration between 1 μM and 100 μM, between 1 μM and 50 μM or between 1 μM and 20 μM, e.g. at a concentration of about 10 μM.

In particular embodiments, the cyclic monophosphate is 2-br-cAMP or 8-br-cAMP, preferably 8-br-cAMP. In particular embodiments, the TGF-β inhibitor is SB431542.

In the methods described herein, the treatment with cyclic monophosphate, and insulin or IGF-1, may last for at last about 14 days, preferably for at least about 21 days, more preferably for at least about 28 days, even more preferably for at least about 36 days. To achieve full maturation of the thyroid organoids, a TGF-beta inhibitor may be added to the culture after at least 14 days, 21 days or 24 days, preferably after at least 28 days of culturing in the presence of cyclic monophosphate, and insulin or IGF-1. Accordingly, in embodiments, the cell culture or the cell population enriched in thyroid and lung progenitor cells is cultured for between 14 and 36 days, preferably for between 21 and 36 days, more preferably for about 28 days, in the presence of cyclic monophosphate, and insulin or IGF-1, and subsequently for at least 5 days or 6 days, preferably for at least 7 days in the presence of a cyclic monophosphate, insulin or IGF-1, and a TGF-β inhibitor. In particular embodiments, the cell culture or the cell population enriched in thyroid and lung progenitor cells is cultured for about 28 days in the presence of cyclic monophosphate, and insulin or IGF-1, and subsequently for at least 7 days in the presence of a cyclic monophosphate, insulin or IGF-1, and a TGF-β inhibitor.

The cell culture or the cell population enriched in thyroid and lung progenitor cells may or may not be expanded prior to inducing differentiation into lung organoids (i.e. prior to culturing in the presence of the cyclic monophosphate, and insulin or IGF-1), e.g. by culturing the cells in a 3D matrix as described elsewhere herein in a basal differentiation medium (without a differentiation inducer) for e.g. between 1 and 14 days such as for between 1 and 7 days. Preferably, the cell culture or the cell population enriched in thyroid and lung progenitor cells is immediately cultured in a differentiation medium (e.g. step d of the methods described herein consists of culturing the thyroid and lung progenitor cells in the 3D matrix in a differentiation medium for a period of time sufficient to generate a lung organoid), which leads to higher efficiency and gene expression and, advantageously, results in a more efficient method.

### Thyroid Organoids

A further aspect relates to thyroid organoids obtainable by the methods described herein. The thyroid organoid is an in vitro or ex vivo generated thyroid organoid, and is preferably a hPSC-derived, in particular a hESC-derived, thyroid organoid.

As shown in the experimental section, during differentiation and maturation in 3D culture the resulting cells form thyroid follicular organoid structures characterized by a monolayered epithelium surrounding a cavity or lumen consistent with thyroid follicles, express genes required for hormone biosynthesis, and can function in vitro and in vivo following transplantation into hypothyroid mice, that is, produce and secrete thyroid hormone. At least 10%, preferably at least 12%, more preferably at least 15%, even more preferably at least 20% or 25% of the cells in the organoid structure express one or more thyroid markers selected from the group consisting of NKX2-1, PAX8, FOXE1, TSHR, TG and TPO.

In embodiments, the thyroid organoids exhibit a thyroid follicular structure comprising a single layer of cells surrounding a lumen.

In embodiments, the thyroid organoids are functional in vitro, i.e. secrete thyroid hormones in vitro. In embodiments, the thyroid organoids are functional in vivo, i.e. secrete thyroid hormones in vivo (e.g. in thyroid gland ablated mice).

In particular embodiments, the thyroid organoids comprise an exogenous nucleic acid sequence encoding NKX2-1 and PAX-8.

Further disclosed herein are a thyroid follicular epithelial cell obtainable by the claimed method and a population of thyroid follicular epithelial cells obtainable by the methods described herein. For example, the thyroid follicular epithelial cells may be isolated from the thyroid organoid structures described herein.

The thyroid follicular epithelial cells as described herein may function like mature thyroid cells, e.g. they may secrete thyroid hormones. In embodiments, the thyroid follicular epithelial cells synthesize and secrete thyroid hormones in vivo. In embodiments, the thyroid follicular epithelial cells synthesize and secrete thyroid hormones in vitro in culture. Advantageously, the thyroid follicular epithelial cells described herein can be grown in culture in vitro and retain the ability to proliferate and retain their thyroid function. In particular embodiments, the thyroid follicular epithelial cells comprise an exogenous nucleic acid sequence encoding NKX2-1 and Pax-8.

In embodiments, the population of thyroid follicular epithelial cells further comprises a cryopreservative for cryostorage. Cryopreservatives or cryoprotective agents and optimal cooling rates can protect against cell injury. Cryoprotective agents which can be used include but are not limited to dimethyl sulfoxide (DMSO), glycerol, polyvinylpyrrolidine, Dextran, trehalose, CryoSoFree (Signa Aldrich Co.) and polyethylene glycol. The preferred cooling rate is 1° to 3° C./minute. After at least two hours, the cells have reached a temperature of −80° C. and can be placed directly into liquid nitrogen (−196° C.) for permanent storage such as in a long-term cryogenic storage vessel.

Yet a further aspect relates to a pharmaceutical composition comprising a thyroid organoid, a thyroid follicular epithelial cell or a population of thyroid follicular epithelial cells as disclosed herein, and a pharmaceutically acceptable carrier.

The term “pharmaceutically acceptable” as used herein is consistent with the art and means compatible with the other ingredients of a pharmaceutical composition and not deleterious to the recipient thereof. As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active substances is well known in the art. Such materials should be non-toxic and should not interfere with the activity of the cells.

Another further aspect relates to a 3-D construct comprising a thyroid follicular epithelial cell or a population of thyroid follicular epithelial cells as disclosed herein, and a matrix or scaffold material wherein the thyroid follicular epithelial cells are embedded. In embodiments, the thyroid follicular epithelial cells are embedded in the matrix or scaffold material and have organized into a 3D organoid structure. The matrix or scaffold material is preferably a biocompatible material. For example, a matrix or scaffold material may be a decellularized tissue such as adipose tissue. In embodiments, the matrix or scaffold material may be a biodegradable synthetic extracellular matrix (sECM).

Cell encapsulation or cell microencapsulation technology involves the immobilization of the desired cells within a polymeric semi-permeable membrane that permits the bidirectional diffusion of molecules such as the influx of oxygen, nutrients, growth factors etc. essential for cell metabolism and the outward diffusion of waste products and therapeutic proteins. At the same time, the semi-permeable nature of the membrane prevents immune cells and antibodies from destroying the encapsulated cells regarding them as foreign invaders. The main motive of cell encapsulation technology is to overcome the existing problem of graft rejection in tissue engineering applications and thus reduce the need for long-term use of immunosuppressive drugs after an organ transplant to control side effects. A variety of biocompatible polymer materials are available and known in the art for cell encapsulation. For examples, alginate, modified alginate having amino acid sequence Arg-Gly-Asp (RGD) for conjugation purposes, alginate-polylysine-alginate (APA), collagen, gelatin, chitosan, agarose and cellulose sulfate to name a few. Hydrogels made with alginate, or collagen, or gelation, or chitosan, or agarose, or cellulose sulfate or combinations of are commonly used. Any method can be used to encapsulate the described population of thyroid follicular epithelial cells.

### Lung Organoids

The lung organoids obtainable by the methods described herein comprise multiple cell types from both the upper airways (such as basal cells, surfactant producing cells (e.g., secretory cells), mucus producing cells (e.g., Goblet cells), ciliated cells and/or neuroendocrine cells) and the lower airways (such as alveolar type 1 and 2 cells).

Basal cells, located in the bronchi and trachea of the human lung epithelium, play a critical role in normal airway homeostasis and repair, and have been implicated in the development of diseases such as cancer. Additionally, basal-like cells contribute to alveolar regeneration and fibrosis following severe injury. Goblet cells secrete mucus, which helps maintain epithelial moisture and traps particulate material and pathogens moving through the airway. The ciliated cells are the primary components in the mucociliary clearance mechanism; their cilia move the mucus-bound particulate up and away for expulsion from the body. Secretory cells have a protective function through secreting a small variety of products, including surfactants. Neuroendocrine cells have an endocrine function. They synthesise, store and release a number of bioactive substances that contribute to redistribution of pulmonary blood flow, regulation of bronchomotor tone, modulation of the immune response, stimulation of sensory nerve fibres and regulation of lung growth and development.

The alveolar epithelial cells line the small, spongy sacs called alveoli that are found throughout the lung. Alveolar type I cells are involved in the process of gas exchange between the alveoli and blood, and alveolar type II cells and alveolar type II cells contribute to lung defense and have regenerative potential.

The different cell types are readily identified by phenotype and/or cell markers known in the art (e.g., by immunofluorescence, histochemical staining, FACS analysis and/or gene expression).

In embodiments, basal cells express one or more markers selected from the group consisting of KRT5 and TP63, preferably TP63.

In embodiments, ciliated cells express one or more markers selected from the group consisting of FOXJ1, TUBB6 and NCS1, preferably FOXJ1.

In embodiments, neuroendocrine cells express one or more markers selected from the group consisting of UCHL1, DLL1 and ASCL1.

In embodiments, secretory cells express one or more markers selected from the group consisting of SOX2, SCGB3a2, MUC1, MUC5AC, MUC5B, CTFR and AGR2, preferably SCGB3a2.

In embodiments, alveolar type 1 cells express one or more markers selected from the group consisting of AKAP5, CLIC5, AQP5, HOPX, PDPN, EMP2, CRYAB and AGER, preferably AGER.

In embodiments, alveolar type 2 cells express one or more markers selected from the group consisting of SFTPB, CXCL5, SLP1, SFTPA1, PGC, CLDN18, ABCA3, LAMP3, SLC34A2, NAPSA and LPCAT1, preferably LAMP3.

The lung organoids described herein are an in vitro or ex vivo generated lung organoid, and are preferably hPSC-derived, more preferably hESC-derived, lung organoids.

### Uses

The thyroid organoids, thyroid follicular epithelial cells and populations of thyroid follicular epithelial cells described herein can be used for transplantation into a subject who is lacking or deficient in thyroid hormones. These cells are also useful as research tools for studying the differentiation signalling pathways involved in thyroid lineage differentiation and thyroid cell maturation, and also for studying the effects of drugs and chemicals on the secretory activities of the mature thyroid cells.

Accordingly, an aspect relates to a thyroid organoid, a thyroid follicular epithelial cell and a population of thyroid follicular epithelial cells as described herein for use in medicine.

A further aspect relates to a thyroid organoid, a thyroid follicular epithelial cell, a population of thyroid follicular epithelial cells or a pharmaceutical composition or a 3D construct comprising said thyroid organoid, said thyroid follicular epithelial cell or said population of thyroid follicular epithelial cells as described herein for use in the treatment of a thyroid hormone insufficiency or deficiency, e.g. hypothyroidism. The organoids or cells can be used for implantation or transplantation into a subject, for example, under the skin, in the neck in the location of the thyroid gland, in muscle tissue, or beneath the kidney capsule.

Hypothyroidism means too little thyroid hormone. Hypothyroidism is a condition in which the body lacks sufficient thyroid hormone. A first common cause of hypothyroidism is a result of previous (or currently ongoing) inflammation of the thyroid gland, which leaves a large percentage of the cells of the thyroid damaged (or dead) and incapable of producing sufficient hormone. The most common cause of thyroid gland failure is called autoimmune thyroiditis (also called Hashimoto's thyroiditis), a form of thyroid inflammation caused by the patient's own immune system. The second major cause is the broad category of “medical treatments”. For example, surgical removal of a portion or all of the thyroid gland to treat a thyroid condition may leave insufficient mass of thyroid producing cells within the body to meet the needs of the body. Also radioactive iodine therapy, e.g. to treat goiters, may damage too many cells so that the patient often becomes hypothyroid within a year or two. Symptoms of hypothyroidism include the following: Fatigue, Weakness, Weight gain or increased difficulty losing weight, coarse, dry hair, dry, rough pale skin, hair loss, cold intolerance compared to those around you, muscle cramps and frequent muscle aches, constipation, depression, irritability, memory loss, abnormal menstrual cycles and decreased libido. Each individual patient may have any number of these symptoms, and they will vary with the severity of the thyroid hormone deficiency and the length of time the body has been deprived of the proper amount of hormone. Most people will have a combination of these symptoms.

A related aspect is directed to a method of treating of a thyroid hormone insufficiency or deficiency, e.g. hypothyroidism, in a subject, said method comprising implanting a thyroid organoid or a 3D construct comprising a thyroid organoid as described herein, or transplanting an effective amount of a thyroid follicular epithelial cell, a population of thyroid follicular epithelial cells or a 3D construct comprising a thyroid follicular epithelial cell or a population of thyroid follicular epithelial cells as described herein into the subject.

The thyroid organoid, the thyroid follicular epithelial cell or the population of thyroid follicular may be autologous or allogenic to the subject. In embodiments wherein the thyroid organoid, the thyroid follicular epithelial cell or the population of thyroid follicular are autologous to the subject, the method may further comprise harvesting a population of pluripotent stem cells from the subject and ex vivo producing a thyroid organoid or a population of thyroid follicular epithelial cells according to a method as described herein for implantation or transplantation.

Yet a related aspect relates to a thyroid organoid, a thyroid follicular epithelial cell or a population of thyroid follicular epithelial cells for use in the manufacture of a medicament for the treatment of a thyroid hormone insufficiency or deficiency, e.g. hypothyroidism.

In embodiments, the subject is a mammal. In embodiments, the subject is a human.

The thyroid organoid described herein can also be used for research purposes. For example, the thyroid organoid can be used to study basic thyroid hormone biogenesis, the emergence of thyroid cancer from follicular precursors, or the molecular pathways that regulate the self-renewal, maturation, proliferation, response to TSH, or longevity of a thyroid cell.

Accordingly, further aspects relate to use of a thyroid organoid as described herein as a model system for studying thyroid development, or as a model system for studying a thyroid disease or disorder.

The thyroid organoid can also be used to screen a library of drugs or small molecules to identify new candidate drugs for treating a thyroid disease or disorder (e.g. autoimmune thyroid diseases, hypothyroidism, hyperthyroidism, or thyroid cancers). Accordingly, a further aspect relates to use of the thyroid organoid as a platform for screening for drugs for the treatment of a thyroid disease or disorder.

A further aspect relates to use of a thyroid organoid as described herein as a platform for testing the effect of a test compound on thyroid tissue, preferably wherein said effect is selected from the group comprising a toxic effect, an effect on thryoid development and a pharmacological effect. A related aspect is directed to a method for studying the effect of a test compound on thyroid tissue, said method comprising: contacting the test compound with a thyroid organoid as described herein, and determining the effect of the test compound on the thyroid organoid, preferably wherein said effect is selected from the group comprising a toxic effect, an effect on lung development and a pharmacological effect.

In embodiments wherein the thyroid organoid is made from the pluripotent stem cells of an individual patient, it can be used as a “precision medicine” tool to predict the individualized responsiveness of that individual to drugs.

The lung organoids described herein are particularly useful for a number of medicinal applications and research purposes.

In embodiments, a lung organoid described herein is used as a model system for studying lung development. For example, the lung organoid may be used to identify the molecular basis of normal human lung development, and/or to identify the molecular basis of congenital defects affecting human lung development.

The lung organoid may also be used to study lung tissue and/or organ responses to radiation, chemotherapy, pharmaceutical therapy, chemical pathogen, toxins, biological exposure, and/or exposure to other environmental stresses. Accordingly, a further aspect relates to the use of the lung organoid described herein as a platform for testing the effect of a test compound or test condition on lung tissue, preferably wherein said effect is a toxic effect or an effect on lung development. A related aspect is directed to a method for studying the effect of a test compound or a test condition (e.g. radiation, environmental stress) on lung tissue, said method comprising:
contacting the test compound with a lung organoid as described herein or exposing a lung organoid as described herein to the test condition, and
determining the effect of the test compound or the test condition on the lung organoid,
   preferably wherein said effect is a toxic effect or an effect on lung development.

In particular embodiments, the lung organoids described herein can be used to screen drugs for lung tissue uptake and mechanisms of transport. Advantageously, this can be done in a high-throughput manner to screen for the most readily absorbed drugs, and can augment Phase 1 clinical trials that are done to study drug lung tissue uptake and lung tissue toxicity.

In other embodiments, the lung organoid is used as a model system for a lung disease or disorder.

The terms “lung disease” and “lung disorder” are used as synonyms for “respiratory disease” and “respiratory disorder”, and “pulmonary disease” and “pulmonary disorder” and refer to any condition and/or disorder relating to respiration and/or the respiratory system, including the lungs, pleural cavity, bronchial tubes, trachea, upper respiratory tract, airways, or other components or structures of the airway system. Non-limiting examples of lung diseases and disorders include bronchopulmonary dysplasia (BPD), chronic obstructive pulmonary disease (COPD), cystic fibrosis, bronchiectasis, cor pulmonale, pneumonia, lung abscess, acute bronchitis, chronic bronchitis, emphysema, pneumonitis (e.g., hypersensitivity pneumonitis or pneumonitis associated with radiation exposure), alveolar lung diseases and interstitial lung diseases, environmental lung disease (e.g., associated with asbestos, fumes or gas exposure), aspiration pneumonia, pulmonary hemorrhage syndromes, amyloidosis, connective tissue diseases, systemic sclerosis, ankylosing spondylitis, pulmonary actinomycosis, pulmonary alveolar proteinosis, pulmonary anthrax, pulmonary edema, pulmonary embolus, pulmonary inflammation, pulmonary histiocytosis X, pulmonary hypertension, surfactant deficiencies, pulmonary hypoplasia, pulmonary neoplasia, pulmonary nocardiosis, pulmonary tuberculosis, pulmonary veno-occlusive disease, rheumatoid lung disease, sarcoidosis, post-pneumonectomy, Wegener's granulomatosis, allergic granulomatosis, granulomatous vasculitides, eosinophilia, asthma and airway hyperreactivity (AHR) (e.g., mild intermittent asthma, mild persistent asthma, moderate persistent asthma, severe persistent asthma, acute asthma, chronic asthma, atopic asthma, allergic asthma or idiosyncratic asthma), allergic bronchopulmonary aspergillosis, chronic sinusitis, pancreatic insufficiency, lung or vascular inflammation, bacterial or viral infections, etc.

For example, the lung organoid described herein may be used to study the development and progression of a lung disease or disorder, as well as to model and generate new therapies for lung diseases and disorders.

In particular embodiments, the lung organoid may be used as a virus infection model, which can be used to study the pathogenic mechanism of viral diseases and/or the defense mechanism of naturally occurring or experimentally induced viral infections, as well as for screening, preferably high-throughput screening, of antiviral drugs and drug development. The virus may be any one selected from SARS-CoV-2 coronavirus, influenza virus, HIV, SARS-CoV coronavirus, MERS-CoV coronavirus, flavivirus, West Nile virus, Chikungunya virus, Mycobacterium tuberculosis, Ebola virus, hepatitis virus, human HPV virus, etc.

The present invention is further illustrated by the following non-limiting examples.

### EXAMPLES

### Example 1: Materials and Methods

Generation of tetracycline-induced hESC line: The human embryonic stem cell line HES3 used in this study was genetically modified at the NKX2-1 locus to allow insertion of sequences encoding green fluorescent protein (GFP), resulting in the NKX2-1^{wt/GFP} hESC line as previously described (Goulburn et al. 2011). To generate an inducible NKX2-1-PAX8 hESC line, the coding sequences of the NKX2-1 and PAX8 genes, separated by a IRES sequence, were cloned into the pInducer20 lentiviral vector (Addgene, Plasmid #44012; RRID: Addgene_44012), which contains the sequences for the TRE/rtTA-inducible system (FIG. 1). Lentiviral supernatants were generated by transient transfection of HEK293 cells according to Lipofectamine™ 2000 (Invitrogen) transfection protocols and harvested 48 h after transfection. To promote integration of sequences into the genome of the NKX2-1^{wt/GFP} HES3 line, hESCs were plated at high density (1:3) in a Matrigel-coated 6-well culture dish and infected with 50 ml of lentivirus supernatant and 6 μg/ml polybrene for 18-20 hours in mTeSR medium (Stem Cell). Positive clones were selected with 300 μg/ml neomycin (Invitrogen). Clones were treated with 1 mg/ml doxycycline (Sigma) for 48 h and screened by immunostaining against NKX2-1 and PAX8 to verify transgene expression. Selected clones were tested for genomic integrity using G-banding technique according to the protocol described by Campos et al. 2009. Journal of Visualized Experiments doi:10.3791/1512).

Pluripotency was assessed by testing the ability of the clones to differentiate into cells from the three germ layers. Cells were cultured in basal differentiation medium (Table 1) for 21 days and the formation of endoderm, mesoderm and ectoderm cells was assessed by immunofluorescence staining against AFP, a-SMA and b-III tubulin, respectively.

TABLE 1
Composition of the basal differentiation medium.

| hESC Differentiation | Stock | Final | Volume | |
|---|---|---|---|---|
| Medium | concentration | concentration | (50 ml) | |
| DMEM/F12 + Glutamax | | | 38.4 | ml |
| FBS | | 20% v/v | 10 | ml |
| MEM-Non-Essential | 100x | 1% | 500 | μl |
| Amino Acids | | | | |
| (MEM-NEAA) | | | | |
| Sodium pyruvate | 100x | 1% | 500 | μl |
| P/S | 100x | 1% | 500 | μl |
| 2-Mercaptoethanol | 7% | 0.007% | 50 | μl |
| Vitamin C | 50 mg/ml | | 50 | μl |

The hESC NKX2-1-PAX8 line was registered and approved by the European Human Pluripotent Stem Cell Registry (hPSCreg) as ESIBIe003-A-6.

hESC culture and differentiation: Modified hESCs were cultured and propagated on Matrigel-coated 6-well culture dishes in Stem Flex medium (Thermo Scientific, A3349401) supplemented with 100 U/ml Penicillin-Streptomycin (Gibco).

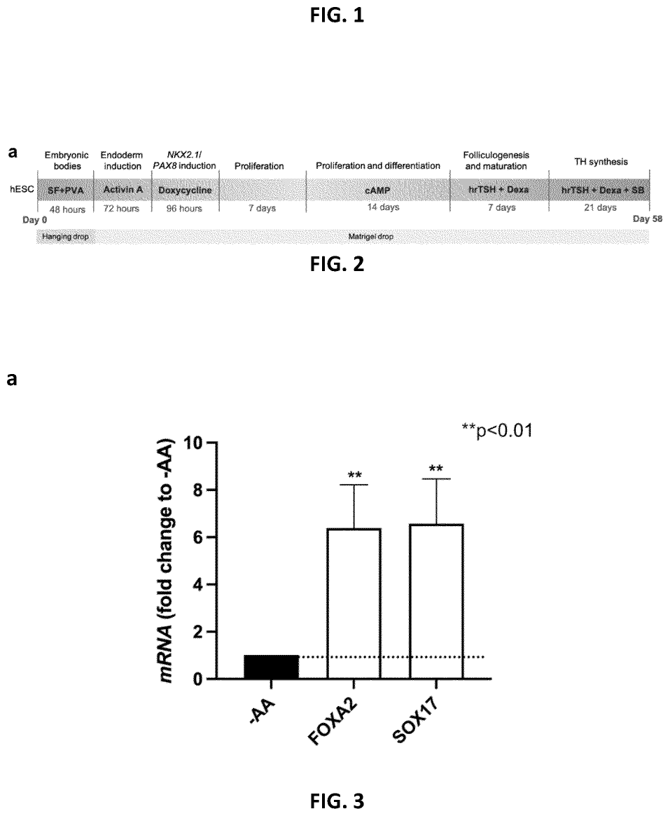

For the generation of embryoid bodies (EBs), highly confluent hESCs were detached with 0.5 mM EDTA solution and diluted with 100,000 cells/ml in Stem Flex medium supplemented with 4 mg/ml polyvinyl alcohol (PVA; Sigma) and EBs formation was induced as previously described (Antonica et al. 2012). Briefly, hESCs (2,000 cells per droplet) were cultured in hanging drops for two days, then EBs were collected and embedded in growth factor-reduced Matrigel (BD Biosciences); 50 μl Matrigel drops (containing approximately 20 embryoid bodies per drop) were replated onto 12-well dishes. Embryoid bodies were differentiated and cultured in differentiation medium containing DMEM/F12+Glutamax (Gibco) with 20% FBS (Gibco), 0.1 mM non-essential amino acids (Gibco), 1 mM sodium pyruvate (Gibco), 0.1 mM 2-mercaptoethanol (Sigma), 100 U/ml Penicillin-Streptomycin (Gibco), and 50 μg/ml L-ascorbic acid (Sigma). Cells were supplemented with 50 ng/ml Activin A (Cell GS) for three days to induce foregut endoderm. Expression of NKX2-1 and PAX8 was induced by incubation with 1 mg/ml doxycycline (Dox; Sigma) for four days. Cells were then cultured in basal differentiation medium for one week to allow expansion of thyroid progenitors, while differentiation and maturation were induced by treatment with 300 μM 8-br-cAMP (Biolog Inc.), 1 mU/ml rhTSH (Genzyme), 50 nM dexamethasone (Dexa; Sigma) and 10 μM SB431542 (Peprotech) where indicated (FIG. 2). Culture medium was changed every 48 hours.

NKX2-1^{GFP+} population assessment—Flow cytometry: hESCs under the thyroid differentiation protocol were collected each week, from day 16 to day 45, and prepared for flow cytometry immunostaining as follows: Matrigel drops (at least 4 samples per time point) were first digested with HBSS solution containing 10 U/ml dispase II (Roche) and 125 U/ml collagenase type IV (Gibco, Thermo Fisher) for 30-60 min at 37° C.; then a single cell suspension was obtained by dissociation with TripLE Express (Thermo Fisher) for 10-15 min incubation at 37° C., the enzymes were inactivated by addition of differentiation medium. After centrifugation, samples were rinsed with PBS and fixed in 1.6% PFA solution in PBS for 15 min at RT, followed by cell permeabilization with 0.1% Triton solution in PBS for 15 min at 4° C. under agitation. After centrifugation, 4% horse serum and 0.5% Tween 20 PBS blocking solution was added for 10 min (4° C. with shaking). The primary anti-rabbit KI67 antibody (1:100) was diluted in the blocking solution and samples were incubated for 30 min (4° C. with shaking). Cells were then rinsed three times with wash solution (0.5% BSA and 0.5% Tween in PBS) and then incubated with Cy5-conjugated anti-rabbit antibody (1:300) diluted in blocking solution for 30 min (4° C. with shaking). NKX2-1^{GFP+} and K67 expression data were obtained and processed using an LSR-Fortessa X-20 flow cytometer and FACSDiva software (BD Biosciences). Unstained cells and isotype controls were included in all experiments. In addition, the percentage of GFP+ cells was used to estimate the thyroid generation efficiency of the protocol.

RNA extraction and quantitative real-time PCR: For total RNA extraction, human organoids (at different time points), in vivo samples, and human thyroid tissue (histologicaly normal thyroid tissue was obtained from a patient undergoing thyroidectomy; Hopital Erasme-ULB Ethics Committee approval; P2016/260), was lysed using RLT lysis buffer supplemented with 1% 2-mercaptoethanol (Sigma), and RNA isolation was performed using the RNeasy micro kit (Qiagen) according to the manufacturer's instructions. For reverse transcription, the Superscript II kit (Invitrogen) was used, and qPCR was performed in triplicates using Takyon (Eurogentec) and CFX Connect Real-Time System (Biorad). Results are presented as linearized values normalized to housekeeping gene, GAPDH (human) or β2-microglobulin (mouse) and the indicated reference value (2-ΔΔCt). Gene expression profile was obtained from at least three independent experiments. Primer sequences are shown in Table 2.

TABLE 2
Primer sequences used for qRT-PCR analysis.

| | Primer Forward | | Primer Reverse | |
|---|---|---|---|---|
| | | SEQ ID | | SEQ ID |
| Gene name | Sequence | NO: | Sequence | NO: |
| FOXA2 | GGGAGCGGTGAAGATGGA | 1 | TCATGTTGCTCACGGAGGAGTA | 2 |
| SOX17 | GTGGACCGCACGGAATTTG | 3 | CACGTCAGGATAGTTGCAGTAAT | 4 |
| *exNXK2-1 | TGTCCTGCTCCACCTTGCT | 5 | CGCACACCGGCCTTATTCCA | 6 |
| *exPAX8 | CCTCGGTGCACATGCTTTAC | 7 | GAGGTCTGCCATTCACAAAGG | 8 |
| NKX2-1 | GCTTCCCCGCCATCTCC | 9 | GCCATGTTCTTGCTCAGCTC | 10 |
| PAX8 | CGAGCGACTCCCCGGCGAT | 11 | GAGGTCTGCCATTCACAA | 12 |
| FOXE1 | GCGACAACCCCAAAAAGTGG | 13 | GCCCAGTAGCCCTTACC | 14 |
| HHEX | GGACGGTGAACGACTACACGCA | 15 | CCAGACGCTTCCTCTCGGCGC | 16 |
| TG | AGACACCTCCTACCTCCCTCA | 17 | TCCTTGGACATCGCTTTGGC | 18 |
| TSHR | TGACCTTTCTTACCCAAGCCA | 19 | TGCTCTCAAGGACTTACACATCA | 20 |
| TPO | CTGTCACGCTGGTTATGGC | 21 | GCTAGAGACACGAGACTCCTCA | 22 |
| NIS/SLC5A5 | ATCGCTATGGCCTCAAGTTCC | 23 | TCCAGGTACTCGTAGGTGCT | 24 |
| GAPDH | CTATAAATTGAGCCCGCAGCC | 25 | TACGACCAAATCCGTTGACTC | 26 |
| Dio1 | ATGGCCAGGAACCCCCG | 37 | GCCTGCTGCCTTGAATGAAA | 38 |

RNA-seq and analysis of bulk samples: Bulk RNA-seq was performed in hESC differentiated cells every week from day 16 to day 45 of the differentiation protocol (FIG. 2). The NKX2-1^{GFP+} cell population was obtained by FACS sorting (FACS Aria; BD Bioscience) after sample preparation was performed as previously described (section “NKX2-1^{GFP+} population expansion assessment—Flow Cytometry”). In brief, 10,000 NKX2-1^{GFP+} cells were directly sorted into 700 μl of Qiazol lysis reagent (Qiagen) and RNA isolation was performed using the miRNeasy micro kit (Qiagen) according to the manufacturer's instructions. RNA concentration and quality were evaluated using Bioanalyser 2100 (Agilent) and RNA 6000 Nano Kit (Agilent). RNA integrity was preserved, and no genomic DNA contamination was detected. Ovarion Solo RNA-seq Systems (NuGen) was used as indicated by the manufacturer, resulting in high-quality indexed cDNA libraries quantified with the Quant-iT PicoGreen kit (Life Sciences) and Infinite F200 Pro plate reader (Tecan); DNA fragment size distribution was examined with the 2100 Bioanalyzer (Agilent) using the DNA 1000 kit (Agilent). Multiplexed libraries (10 pM) were loaded onto flow cells and sequenced on the HiSeq 1500 system (Illumina) in high-output mode using the HiSeq Cluster Kit v4 (Illumina). Approximately 10 million paired-end reads were obtained per sample. After removal of low quality bases and Illumina adapter sequences using Trimmomatic software (Bolger et al. 2014. Trimmomatic: a flexible trimmer for Illumina sequence data. Bioinformatics 30), sequence reads were aligned against the human reference genome (Hg19) using HiSat2 software (Kim et al. 2015. HISAT: a fast spliced aligner with low memory requirements. Nature methods 12). Raw reads were determined with HTSeq software (Anders et al. 2015. HTSeq—a Python framework to work with high-throughput sequencing data. Bioinformatics 31) using the Ensembl genome annotation GRCh38.p13. Normalization and differential expression analyzes were performed with two biological replicates per sample using the website iDEP version 0.93 (Ge et al. 2018. iDEP: an integrated web application for differential expression and pathway analysis of RNA-Seq data. BMC Bioinformatics 19). Genes for which expression values were lower than 5 were filtered out. The fold changes of mean gene expression for the duplicates were used to calculate the level of differential gene expression.

Single cell RNAseq characterization of thyroid organoids: Cells originating from human thyroid differentiation protocol, at day 45, were isolated for scRNAseq profiling, following the procedures described in Romitti et al. (2021. Single-Cell Trajectory Inference Guided Enhancement of Thyroid Maturation In Vitro Using TGF-Beta Inhibition. Frontiers in Endocrinology 12). Single cell suspension preparation and FACS cell sorting were performed as described elsewhere herein (“Cell proliferation assessment—Flow cytometry” and RNA-seq and analysis of bulk samples sections). Different proportions of viable NKX2-1/GFP+ (60%) and NKX2-1/GFP−(40%) cells were sorted to guarantee representation of the distinct cell types present in the organoid culture. Sorted cells were collected in PBS at a density of 800 cells/ul and diluted accordingly to kit's instruction (10x Genomics Chromium Single Cell 3′ v3). Around 6,000 cells were loaded onto a channel of the Chromium Single Cell 3′ microfluidic chip and barcoded with a 10X Chromium controller followed by RNA reverse transcription and amplification according to manufacturer's recommendations (10X Genomics). Library preparation was performed based on 10x Genomics guidelines. Libraries were sequenced using Illumina NovaSeq 6000 system.

Single cell RNAseg data analysis: Raw sequencing data was aligned, annotated, demultiplexed and filtered using Cell Ranger Software (v.6.0.1) with a custom-built reference. The custom-built reference was based on the human reference genome GRCh38 and gene annotation Ensembl 98 in which the EGFP sequence was included. The new reference was generated using the cellranger mkref function from the Cell Ranger Software. Analyses were done using R 4.1.0 and Seurat version 4.0.3 47. Briefly, raw counts from Cell Ranger were loaded and the “background soup” was removed using SoupX 48. The background soup refers to ambient RNA molecules contaminating cell-containing droplets, a common problem in droplet-based single cell RNA-sequencing technologies. Decontaminated UMIs were then filtered to discard any doublet (droplet containing two cells instead of 1) using DoubletFinder (McGinnis et al. 2019 DoubletFinder: Doublet Detection in Single-Cell RNA Sequencing Data Using Artificial Nearest Neighbors. Cell Systems 8). Finally, cells containing less than 200 unique genes or more than 26% of UMI counts related to mitochondrial genes were discarded. The 26% threshold was selected to discard dying cells while retaining as much barcodes as possible. The resulting library was scaled and normalized using the SCTransform function from Seurat. Cell cycle effects and mitochondrial content were used as variables to regress out with SCTransform. Principal component analysis (PCA) was computed using the 3000 first variable features, and the top 30 principal components were used for SNN graph construction, clustering (resolution 1) and UMAP embedding using Seurat's functions and recommended methods. Cluster annotation was based on marker genes obtained using Seurat's FindAllMarkers function and literature survey. Pseudotime analysis in thyroid populations was performed using Monocle3 (Cao et al. 2019. The single-cell transcriptional landscape of mammalian organogenesis. Nature566:496-502) with default parameters and with data imported from the Seurat object, selecting thyroid progenitors as root cells. Pseudotime-related plots were generated using the FeaturePlot function from Seurat and the geom_smooth function from ggplot2. Receptor-ligand interaction analysis was done with CellPhoneDB, which consists in a public repository of ligands, receptors and their interactions enabling a comprehensive and systematic analysis of cell-cell communication (Efremova et al. 2020 CellPhoneDB: inferring cell-cell communication from combined expression of multi-subunit ligand-receptor complexes. Nature Protocols 15). CellphoneDB was run using the statistical method with default parameters. A manually selected list of biologically relevant ligand receptor pairs displaying statistically significant interaction was used to create the dot plot showing the interactions of thyroid populations with other cell populations.

Follicles enrichment for in vivo transplantation: Thyroid organoids at day 45 of differentiation were washed twice with Hanks's balanced salt solution (HBSS, containing calcium and magnesium; Gibco), then 1 ml of a digestion medium containing 10 U ml dispase II (Roche) and 125 U ml of collagenase type IV (Sigma) diluted in HBSS was added to each well. The organoids were carefully removed using a 5 ml pipette and transferred to a sterile Erlenmeyer and incubated at 37° C. in a water bath with shaking for 45-60 min. The release of thyroid follicles was tracked by microscopy (bright field and GFP). When isolated structures were detected, enzymes were inactivated by addition of 10% FBS followed by centrifugation at 500 g for 3 min. Cells were rinsed twice with HBSS and the follicles population was enriched using 30 μm (single cell removal) and 100 μm (follicles enrichment; 30-100 μm size) reverse strainer (Pluriselect). Finally, the 3D-structures were counted and approximately 10,000 structures were resuspended in 65 μl of differentiation medium for in vivo transplantation.

RAI-induced hypothyroidism mouse generation, transplantation of hESC-derived thyroid follicles and SPECT-CT imaging: All animal experiments were performed in accordance with local Animal Ethics (Ethical Project CMMI-2020-01). A cohort of 20 five week-old female non-obese and non-diabetic mice with severe combined immunodeficiency (NOD-SCID) (Charles River Laboratories, France) was placed on an iodine-deficient diet for one week after arrival. In addition, six NOD-SCID mice, not submitted to any treatment were included in the study as external controls. One week after starting the diet (first week), 14 of the 20 mice were injected intraperitoneally with approximately 5.75 MBq (90 μL supplemented with 10 μl NaCl 0.9% solution (MiniPlasco, BBraun) Iodide ¹³¹I-Injection-IBS.2P (GE Healthcare Belux, Belgium). To confirm the destruction of functional thyroid tissue by ¹³¹I injection, SPECT-CT images of Sodium Iodide ¹²³I uptake were obtained on a nanoSPECTPlus (for the SPECT) and a nanoScanPETCT (for the CT) (Mediso, Hungary) equipped with a Minerve rat cell implemented with a mouse insert. In the fourth week, the 20 mice were injected intravenously with 8.75-9.33 MBq ¹²³I 24 hours before imaging. SPECT/CT imaging was performed on two mice in parallel under isoflurane anesthesia (1.8% isoflurane, 2.0 l/min O2) with the following parameters: collimator aperture APT105, ‘fast’ helicoidal acquisition mode with a duration of 50 s/projection to acquire 1000 counts per projection, scan range of 105 mm, reconstruction in standard mode, i.e. 35% smoothing, 3 iterations and 3 subsets to obtain a voxel size of 750 μm³. CT was performed with the following parameters: 480 projections, minimum zoom, binning 1:4, 50 kV, 300 ms/proj, scan range of 115 mm. Acquisition data were reconstructed with a Feldkamp-based algorithm generated to obtain a cubic voxel of 250 um³, using a cosine filter with a cut-off of 100%. Then, one week later (week five) 6 irradiated mice were transplanted with thyroid organoids. First, control and thyroid gland ablated mice were treated with 0.01 mg/ml-50 μl Temgesic (Schering Plow), anesthetized under isoflurane anesthesia, and the eyes/cornea were protected with Vidisic gel (Bausch & Lomb Inc.). Mice were injected with 8 μl of follicle-enriched suspension thyroid organoids (described in “Enrichment of follicles for in vivo transplantation”) into the unilateral kidney under the capsule using a 30G needle syringe (Hamilton Bonaduz AG) (the kidney was exposed through skin/muscle/peritoneum incision via a dorsolateral approach). The entire cohort of mice was imaged 4 weeks after transplantation (week 9) as described above to assess the iodine uptake capacity of the transplanted tissue. Due to the radiosensitivity of immunodeficient mice, 30% of the irradiated animals died during the experimental period. At the end of the experiment (week ten), 6 non-transplanted and 4 transplanted mice had survived and could be analyzed. Mice were finally sacrificed, blood collection was performed for the T4 assay, while the kidney and transplanted tissues were harvested for transcriptomic and histological analyzes. Qualitative and quantitative analysis of the images was performed using VivoQuant v3.5 software (InVicro, USA). Radioiodine uptake in thyroid tissue and/or graft was evaluated according to the design of volumes of interest (VOI) based on the corresponding radioactive signal. The % injected dose (% ID) was calculated as previously described by Brandt, et al. (2012. Micro-single-photon emission computed tomography image acquisition and quantification of sodium-iodide symporter-mediated radionuclide accumulation in mouse thyroid and salivary glands. Thyroid 22:617-624) and results were expressed as % ID/organ.

Plasma T4 measurement: Total T4 levels were measured by Mouse/Rat T4 Total ELISA kit (T4044T-100 Calbiotech) according to the manufacturer's instructions.

Plasma T3 measurement: T3 levels were measured in 50 μl of plasma after extraction with chloroform-methanol 2:1 containing 1 mM propylthiouracil prior to measurement by highly sensitive radioimmunoassay, using an antibody produced by Gabriela Morreale de Escobar as described in a supplement by Ferrara et al. (2013. Endocrinology 154:2533-2541). The limit of detection was 5 ng/dL.

TSH measurement: TSH was measured in 50 μl of plasma using a sensitive, heterologous, disequilibrium, double-antibody precipitation radioimmunoassay. Due to the expected high concentrations of TSH in serum of RAI-treated mice, proper dilutions were made using TSH null mouse serum. The limit of detection was 10 mU/L.

Immunofluorescence staining: For immunofluorescence staining, cells cultured in monolayer or MTG-drop were fixed in 4% paraformaldehyde (PFA; Sigma) for 2 h at RT, washed three times in PBS, and blocked in 3% bovine serum albumin (BSA; Sigma), 5% horse serum (Invitrogen), and 0.3% Triton X-100 (Sigma) PBS solution for 30 min at room temperature. Primary and secondary antibodies were diluted in a PBS solution of 3% BSA, 1% horse serum, and 0.1% Triton X-100. Primary antibodies were incubated overnight at 4° C., then washed three times and incubated with secondary antibodies for 2 h at room temperature. The nuclei were stained with Hoechst 33342 (Invitrogen). The slides were mounted with Glycergel (Dako). For paraffin embedding, in vitro organoids and grafted samples were fixed overnight at 4° C. in 4% PFA and kept in 70% ethanol at 4° C. for at least 24 hours at 4° C. before embedding. Samples were then embedded in paraffin, sectioned (5 μm), mounted on glass slides, deparaffinized, and rehydrated. For histological analysis, sections were stained with hematoxylin and eosin (H&E) according to a routine protocol. For immunostaining, antigen retrieval was performed by incubating the sections for 10 min in the microwave (850 W) in Sodium Citrate Buffer (10 mM Sodium Citrate, 0.05% Tween 20, pH 6.0). After cooling, the sections were rinsed with PBS and then blocked with 1% BSA and 10% horse serum PBS solution for 1 h at RT. Primary antibodies were diluted in the blocking solution and incubated overnight at 4° C. The sections were rinsed three times in PBS and incubated with Hoechst 33342 (Invitrogen) and secondary antibodies diluted in blocking solution for 1 h at room temperature. Slides were mounted with Glycergel (Dako). Information on antibodies and sources are listed in Table 3.

TABLE 3
Primary and secondary antibodies used.

| | | | | | |
|---|---|---|---|---|---|
| Protein | | Catalog | Host | Dilution | Dilution |
| Target | Supplier | number | Species | Flow Cyt. | IF |

Primary antibodies

| | | | | | |
|---|---|---|---|---|---|
| AFP | Santa Cruz | sc-8108 | Goat | | 1:100 |
| β-III | Eurogentec | MMS-435P- | Mouse | | 1:1,000 |
| Tubulin | | 200 | | | |
| αSMA | Abcam | ab32575 | Rabbit | | 1:1,000 |
| NKX2-1 | Abcam | ab76013 | Rabbit | | 1:500 |
| PAX8 | Cell Signaling | 59019 | Rabbit | | 1:500 |
| TG | Dako | A0251 | Rabbit | | 1:2,000 |
| TG | Abcam | Ab187378 | Mouse | | 1:250 |
| TPO | Santa Cruz | sc-58432 | Mouse | | 1:100 |
| T4 | Biorbyt | orb11479 | Goat | | 1:1,000 |
| T4 | Invitrogen | MA5-14716 | Mouse | | 1:100 |
| E-cadherin | BD | 610181 | Mouse | | 1:1,000 |
| CD31 | R&D | AF3628 | Goat | | 1:100 |
| HNA | Abcam | ab190710 | Mouse | | 1:250 |
| KI67 | Abcam | ab15580 | Rabbit | 1:100 | |

Secondary antibodies

| | | | | | |
|---|---|---|---|---|---|
| Cy3- | Jackson | 715-165-150 | Donkey anti- | | 1:500 |
| conjugated | Immunoresearch | | mouse IgG | | |
| Cy3- | Jackson | 711-165-152 | Donkey anti- | | 1:500 |
| conjugated | Immunoresearch | | rabbit IgG | | |
| Cy3- | Jackson | 705-165-147 | Donkey anti- | | 1:500 |
| conjugated | Immunoresearch | | goat IgG | | |
| Alexa fluor | Jackson | 715-545-150 | Donkey anti- | | 1:500 |
| 488- | Immunoresearch | | mouse IgG | | |
| conjugated | | | | | |
| Alexa fluor | Jackson | 715-605-150 | Donkey anti- | | 1:500 |
| 647- | Immunoresearch | | mouse IgG | | |
| conjugated | | | | | |
| Alexa fluor | Jackson | 711-605-152 | Donkey anti- | 1:300 | 1:500 |
| 647- | Immunoresearch | | rabbit IgG | | |
| conjugated | | | | | |

Imaging: Fluorescence imaging was performed on a Zeiss LSM510 META confocal microscope, a Zeiss Axio Observer Z1 microscope with AxioCamMR3 camera, and a Leica DMI6000 with DFC365FX camera. Hematoxylin and eosin whole slide images were acquired using a NanoZoomer-SQ digital slide scanner C13140-01 (Hamamatsu) and images were generated using NDP.view 2 software (Hamamatsu).

Statistical analysis: Statistical significance between two groups was tested using the unpaired t test or the nonparametric Mann-Whitney U test, while comparison between multiple groups was performed using one-way ANOVA or Kruskal-Wallis tests. Data are presented as mean± SD or median (IQR). Differences were considered significant at p<0.05. GraphPad Prism version 9 was used for most analyses (GraphPad Software). Data presented are from at least three independent experiments.

### Data Availability

Bulk RNA-seq and Single-cell RNA-seq data have been deposited in the NCBI Gene Expression Omnibus under accession number GSE181452 and GSE181256, respectively.

### Example 2: In Vitro Differentiation of Human Thyroid Cells

hESC Line Generation and Characterization

A previously generated NKX2-1^{WT/GFP} knock-in hESC line to track thyroid differentiation and cell organization using the NKX2-1^{GFP} reporter (Goulburn et al. 2011. Stem Cells 29:462-473) was modified to allow transient expression of NKX2-1 and PAX8, by adding doxycycline (Dox; 1 mg/ml; FIG. 1) to the culture medium (FIG. 2). The resulting hESCs, had normal karyotype and ability to spontaneously differentiate into cells from the three germ layers, i.e. ectoderm, endoderm and mesoderm cells.

### Induction of Thyroid Status

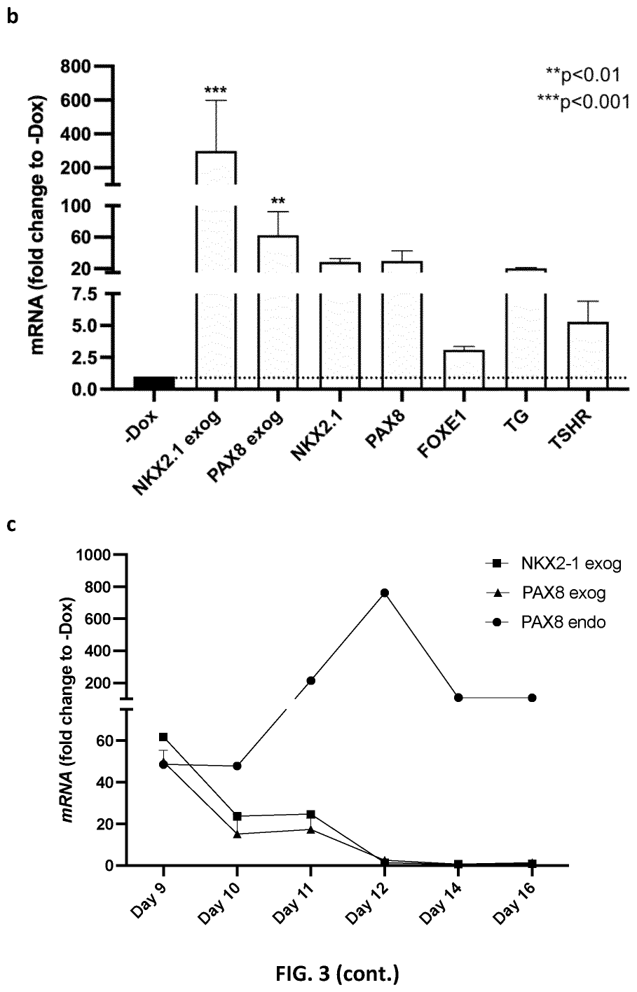

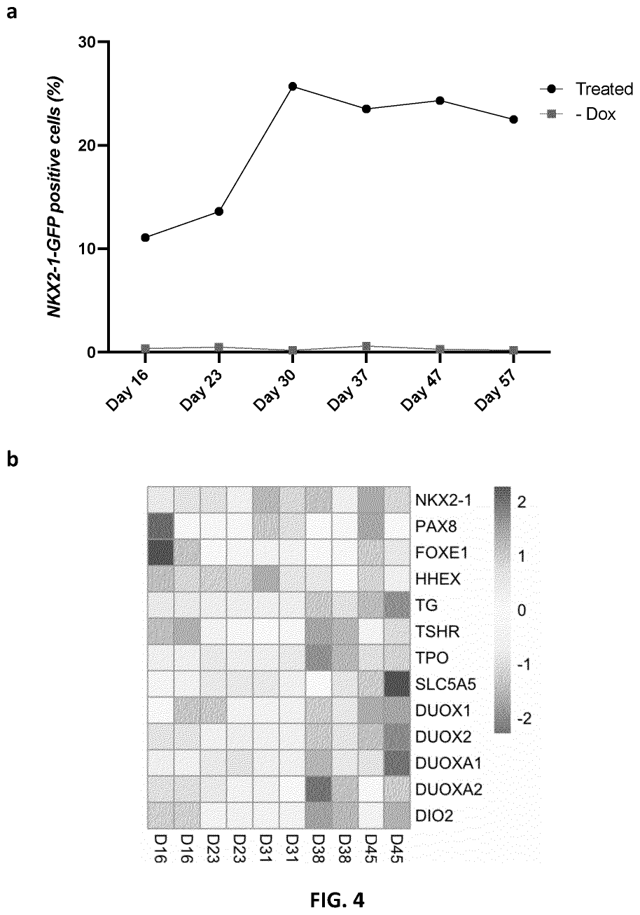

The modified hESCs were first grown for 2 days in hanging drops to allow the formation of embryoid bodies (EBs) (FIG. 2). The generated EBs were then cultured in matrigel drops and endoderm was induced by adding Activin A for 3 days. This treatment resulted in increased mRNA levels of the endoderm markers SOX17 and FOXA2 (FIG. 3a) and simultaneously improved the percentage of FOXA2+ cells, particularly in the inner compartment of EBs (data not shown).

After induction of the endoderm, Dox treatment promoted the overexpression of NKX2-1 and PAX8. After 4 days, expression of NKX2-1 and PAX8 was detected by immunofluorescence in a large proportion of Dox-treated cells but not in the absence of Dox (data not shown). Furthermore, qPCR analysis showed that not only were exogenous NKX2-1 and PAX8 gene expression levels significantly upregulated, but endogenous NKX2-1, PAX8, FOXE1, TG, and TSHR mRNA levels were also increased as early as day 9 (FIG. 3b). To determine whether forced overexpression of thyroid TFs leads to autonomous activation of endogenous cell programming in thyroid fate, Dox treatment was interrupted and cells were incubated in basal differentiation medium for 7 days (from day 9 to day 16). qPCR analysis revealed that exogenous PAX8 expression at day 9 was similar to endogenous PAX8 expression, but the levels of exogenous TFs decreased over time and reached control levels (+AA −Dox) from day 12. In contrast, endogenous PAX8 levels increased dramatically and reached a plateau from day 14 (FIG. 3c). These results suggest that Dox induction of TFs the endogenous transcriptional machinery is activated, initiating the thyroid differentiation program.

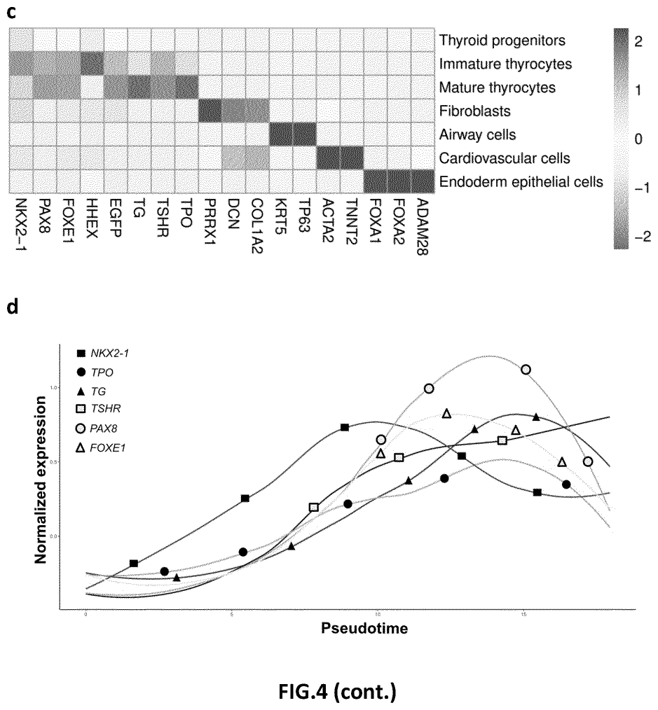

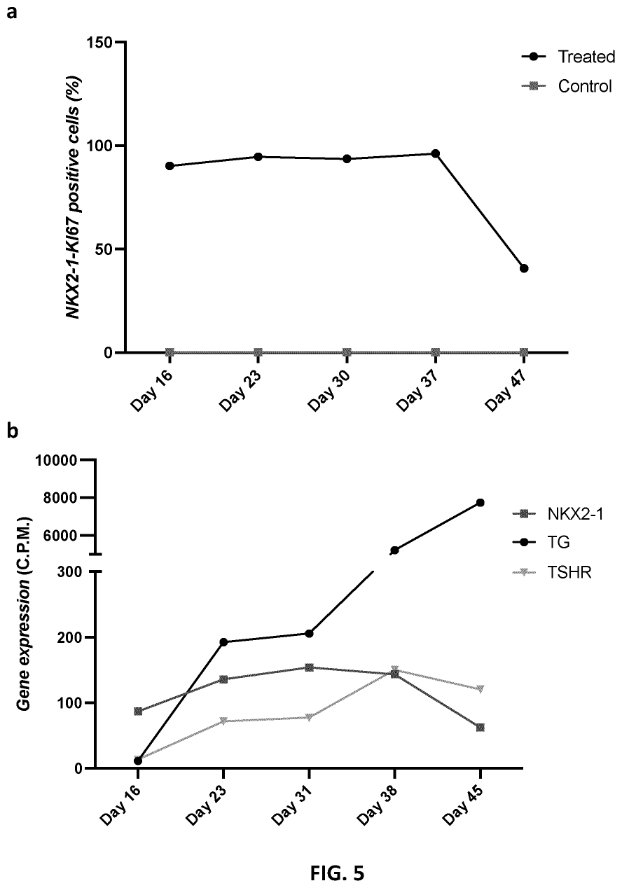

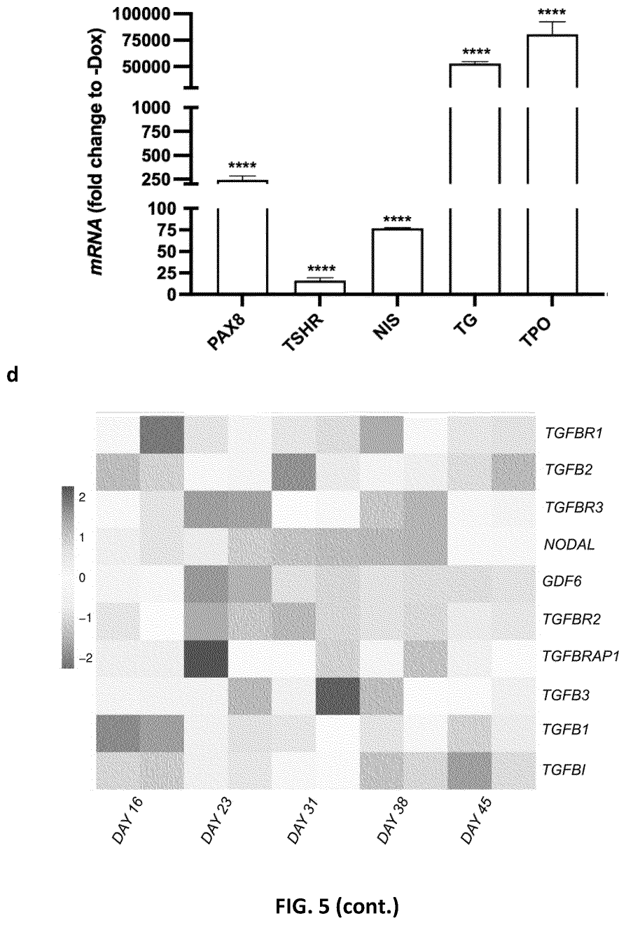

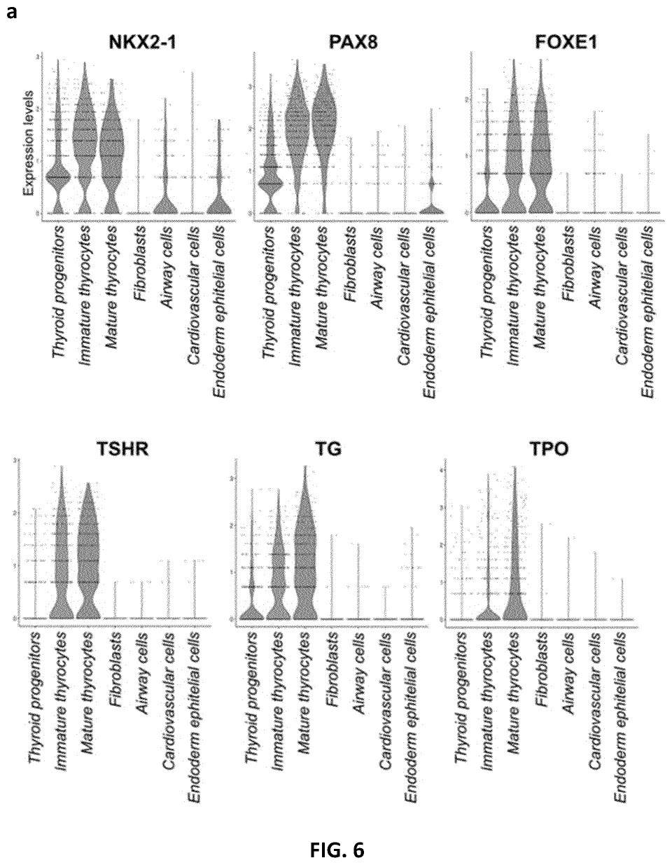

Thyroid cell population expansion and early differentiation were promoted by incubation with 8-br-cAMP for 2 weeks (from day 16 to day 30) (FIG. 2). Flow cytometry analysis confirmed growth of the thyroid population resulting in approximately 25% of total cells expressing NKX2-1^{GFP} at day 30 (FIG. 4a), reflecting the increase in proliferation as around 90% of NKX2-1^{GFP+} cells continuously expressed KI67 during the treatment period (FIG. 5a). Accordingly, transcriptomics analysis performed in NKX2-1GFP cells showed high levels of various proliferation markers (data not shown). In parallel, an increase in expression of early thyroid markers was also observed over time (+AA +Dox +cAMP) (Fig. 4b). This was accompanied by a steady expression of key genes such as NKX2-1, TG, and TSHR from day 23 (FIG. 5b). However, key maturation markers, such as NIS, TPO and DUOX family, were not significantly induced by cAMP, suggesting that it is not sufficient to promote thyroid maturation and function. By tracking NKX2-1^{GFP+} cells we observed at day 28, that thyroid cells start to form follicle-like structures and immunostaining shows marked expression of TG and PAX8. Though, the cells were not organized in single-layered follicles, but a luminal compartment was observed (data not shown), suggesting that the process of folliculogenesis is not yet complete at this stage.

Thyroid maturation and function: since cAMP treatment was not able to fully promote thyroid maturation despite significant expression of TSHR, we first replaced cAMP with hrTSH from day 30. Second, we added dexamethasone (from day 30) and the TGFβ inhibitor SB431542 (from day 37), based on transcriptomic data showing substantial levels of TGFβ pathway markers (Fig. 5d) among NKX2-1 cells. Together, these alterations resulted in significant improvement in the expression of key thyroid maturation markers, including TSHR, TG, NIS/SLC5A5, TPO, DIO2, and the DUOX family (FIG. 4c and FIG. 5c), while mRNA levels of TGFβ pathway effectors were reduced, particularly receptors (FIG. 5d). Subsequently, PAX8 and ZO-1 co-staining revealed monolayer-organized follicles with a well delimited lumen (data not shown). On the other hand, the proportion of NKX2-1+ cells was maintained over time, whereas NKX2-1/KI67+ cells clearly decreased at day 47, compared to the early time points (FIG. 4a and FIG. 5a, respectively).

This human hESC-derived protocol for thyroid generation followed the sequential events observed in vivo, as in human, thyroid development takes approximately 40 days from specification to folliculogenesis (Fernández et al. 2015. Nature Reviews Endocrinology 11:29-42; Dom et al. 2021. Frontiers in Cell and Developmental Biology 9), a similar developmental time was required in the present in vitro model. In addition, following forced expression of NKX2-1 and PAX8, the expression of maturation genes followed the physiological sequence, with TG and TSHR being the first detected genes, followed by TPO and NIS/SLC5A5 (Fernández et al. 2015. Nature Reviews Endocrinology 11:29-42; Dom et al. 2021. Frontiers in Cell and Developmental Biology 9). A similar effect trend was observed in thyroid population expansion, organization and follicle formation. However, even though the TH machinery seems to be complete, TH-producing follicles were not detected at day 45.

### Complete In Vitro Human Thyroid Maturation and Thyroid Hormone Synthesis

To promote functionality, the organoids were kept in culture for two additional weeks using the same conditioned medium (FIG. 2).

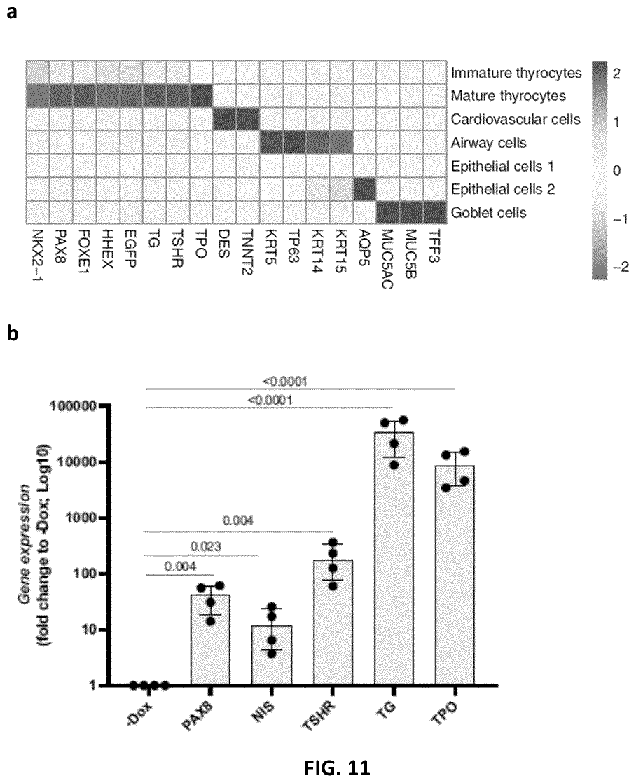

Consistent with the improvement inmaturation detected by scRNAseq at day 58 (Example 3), RNA expression measured by qRT-PCR confirmed the maintenance of thyroid gene expression levels with a marked increase of TSHR mRNA compared to day 45 (FIG. 11b).

Immunostaining revealed a large proportion of well-organized follicles expressing NKX2-1, E-CADHERIN (data not shown) and TG which accumulated mainly in the lumen (data not shown). In addition, marked TPO staining was observed in most follicular structures, with accumulation at the apical membrane indicating an enhancement of maturation (data not shown).

Finally, prolongation of the differentiation protocol resulted in TH synthesis, as evidenced by the detection of T4 in the lumen of hESC-derived thyroid follicles (data not shown).

### Example 3: Single Cell Characterization of Human Thyroid Organoids

To characterize the resulting cell composition of the hESC-derived thyroid model obtained in Example 2, scRNA-seq analyzes were performed at day 45 and at day 58.

Since the efficiency of the protocol was approximately 25%, the proportion of NKX2-1^{GFP+} cells was enriched to 60%, the remaining sorted cells belonging to the GFP-population. A total of approximately 6,000 cells were used for scRNA-seq library preparation using the droplet-based assay from 10X Genomics. After quality control, 1874 cells were obtained that met all the criteria.

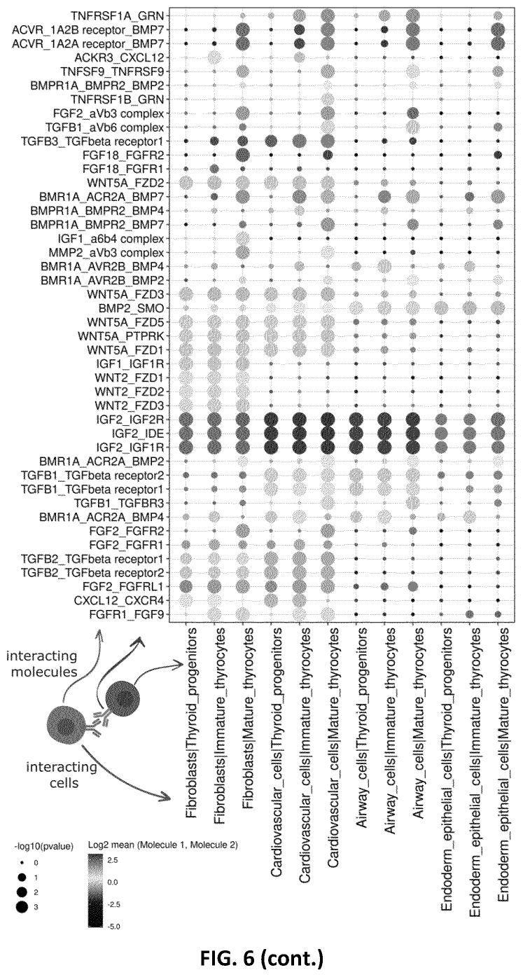

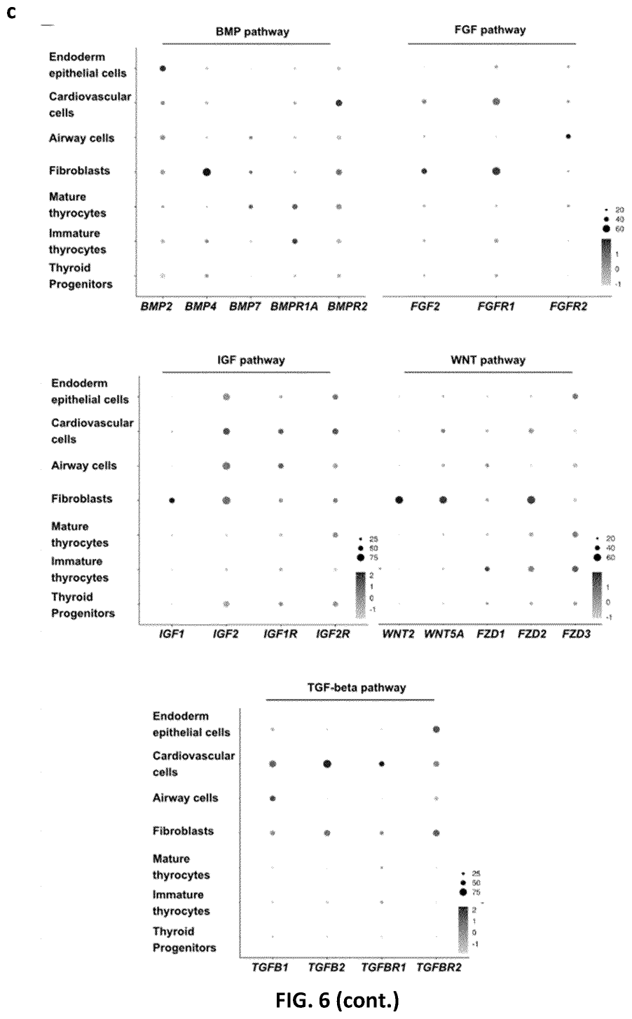

Among those cells, 7 clusters were identified (FIG. 4c), including a cluster of thyroid follicular cells with 1176 cells showing expression of genes involved in development and function, including NKX2-1, PAX8, FOXE1, HHEX, TG, TSHR, and TPO (FIG. 4c). Three sub-clusters were identified among the thyroid cells: Thyroid “progenitors” (477 cells) expressing mainly the thyroid TFs; immature thyrocytes (365 cells) expressing also TG and TSHR; and mature thyrocytes (334 cells) showing a canonical thyroid signature with a higher proportion of TPO-expressing cells (FIG. 4c and FIG. 6a). Furthermore, pseudotime analysis revealed a branch of differentiation originating from thyroid progenitors and moving toward immature and progressive mature thyrocytes (data not shown). This transition was used to generate the trend of gene expression for the different thyroid markers along the trajectory (FIG. 4d), with TFs appearing first, followed by TSHR, TG, and TPO expression (FIG. 4c).

The identity and molecular signature of the remaining cells was characterized. Four non-thyroidal clusters were identified: fibroblasts (146 cells) expressing DCN, COL1A2, and PPRX1; cardiovascular cells (182 cells) enriched in ACTA2 and TNNT2 markers; airway cells (203 cells) expressing KRT5 and TP63 and endoderm-epithelial cells (167) expressing FOXA1, FOXA2, and ADAM28 (FIG. 4c).

Even if NKX2-1 also plays a critical role in lung and forebrain development (Lazzaro et al. 1991. The transcription factor TTF-1 is expressed at the onset of thyroid and lung morphogenesis and in restricted regions of the foetal brain. Development vol. 113), the protocol as described in Example 2 predominantly generated thyroid cells, since more than 75% of NKX2-1+ cells co-expressed PAX8 and/or other thyroid markers.

Connectome: To predict possible crosstalk between thyroid cells and the other cells present in the organoids, CellPhone-DB was used to access the ligand-receptor interaction pairs identified between thyroid clusters and other cell types. Significant cell-cell interactions were found between thyroid cells with mainly mesodermal cells associated with several signalling pathways described as involved in thyroid development and physiology (FIG. 6b). In paricular, it was observed that the presence of mesoderm-derived cells could be beneficial for thyroid development without supplementation of factors, as fibroblasts and cardiovascular cells were an important source of BMP2, BMP4, and FGF2 ligands, whereas thyroid cells expressed the specific receptors (FIG. 6b-c). In addition to BMP and FGF, mesodermal, airway and endoderm epithelial cells also provided significant amounts of IGF-1 and IGF-2, while progenitors and immature thyrocytes expressed IGF1R and mature thyrocytes mainly expressed IGF2R. On the other hand, fibroblasts and cardiovascular cells expressed significant amounts of WNT2, WNT5A, TGFb1, and TGFb2, whereas thyroid cells expressed their respective receptors (FIG. 6b-c).

As described in Example 2, inhibition of TGFβ signaling lead to enhanced thyroid maturation, and, without wishing to be bound by any theory, this effect may also be related to repression of such signals from mesodermal-like cells.

Despite the cell differentiation and follicular organization observed at day 45, single-cell RNA profiling revealed that a substantial proportion of the thyrocyte population was not fully mature. Without wishing to be bound by any theory, this may explain the lack of TH detection at this stage. To promote functionality in vitro, the thyroid cells were kept in culture. Based on the time required for thyroid full maturation and TH synthesis in vivo, the organoids were kept for two additional weeks in the same conditioned medium (FIG. 2). Another scRNAseq transcriptomic analysis was performed at day 58, following the same protocol steps and conditions as for day 45.

At day 58, 2386 cells met the quality control criteria. 7 clusters were identified (FIG. 11a), including a cluster of thyroid follicular cells with 788 cells showing expression of genes involved in thyroid development and function. Unlike day 45, only two thyroid cells subclusters were identified: immature thyrocytes (560 cells) co-expressing the four TFs (NKX2-1, PAX8, FOXE1, HHEX), TG and TSHR; and mature thyrocytes (228 cells) showing a canonical thyroid signature with TPO-expressing cells (FIG. 11a). Integration analysis was used to evaluate similarity between the scRNAseq datasets from day 45 and 58. The analysis revealed a high degree of overlap between immature and mature thyroid cells, and between cardiovascular, airway and goblet cells from the two datasets. Furthermore, the similarity of the day 45 and 58 organoids were compared with a dataset of human adult thyroid tissue from the Human Cell Landscape: cells of thyroid organoids and human thyroid tissue clearly overlapped.

The identity and molecular signature of non-target cells were characterized. Five non-thyroidal clusters were found, 3 of which were already identified at day 45: cardiovascular cells (149 cells) enriched in DES and TNNT2 markers; airway cells (430 cells) expressing KRT5 and TP63 and goblet cells (293) expressing MUC5AC, MUC5B and TFF3 (FIG. 3b). Two new clusters that did not show specific signatures of any known cell type were identified. Based on the expression of epithelial markers, such as KRT14 and KRT7, they were labeled “epithelial cells 1” and “epithelial cells 2”. The later also expressed AQP5 (FIG. 3b). Immunostaining confirmed the presence of these cell populations in the thyroid organoid system (data not shown).

Of note, the proportion of thyroid cells identified at day 58 was lower than at day 45. Without wishing to be bound, this may be due to the low proliferation rate of thyroid follicles compared with lung cells (airway and goblet cells population). Also, and without wishing to be bound by any theory, the lack of a defined cluster of fibroblasts might be due to the long treatment with SB431542, as the TGFβ signaling pathway plays an important role in fibroblast proliferation and maintenance.

Connectome: CellPhone-DB revealed fewer ligand-receptor interactions between follicular and other cell clusters at day 58 compared with day 45. Significant cell-cell interactions were observed between thyroid and cardiovascular cells associated with several signaling pathways such as TGFβ, BMP, FGF, WNT and IGF. To a lesser extent, airway and goblet cells appeared to interact with thyroid cells by producing mainly IGF ligands, whereas these cell populations appeared to interact only with immature thyrocytes via BMP signaling. On the other hand, only airway cells showed expression of WNT ligands interacting with thyroid cells expressing the cognate receptors. The analysis revealed no significant interactions involving epithelial cells 1 and 2. As for day 45, immunostaining for TG and Troponin T (a marker for cardiomyocytes) showed that the thyroid population was in close proximity to the cardiovascular cells supporting a possible interaction between these cells.

### Example 4: Assessment of In Vivo Functionality of hESC-Derived Thyroid Follicles

To evaluate the in vivo functionality of hESC-derived thyroid follicles obtained in Example 2, the recovery of TH was measured in NOD-SCID mice whose thyroid gland was ablated with radioactive iodine (RAI) following low iodine diet to enhance thyroidal RAI uptake. Thyroid ablation was confirmed after 4 weeks by SPEC-CT imaging with ¹²³I.

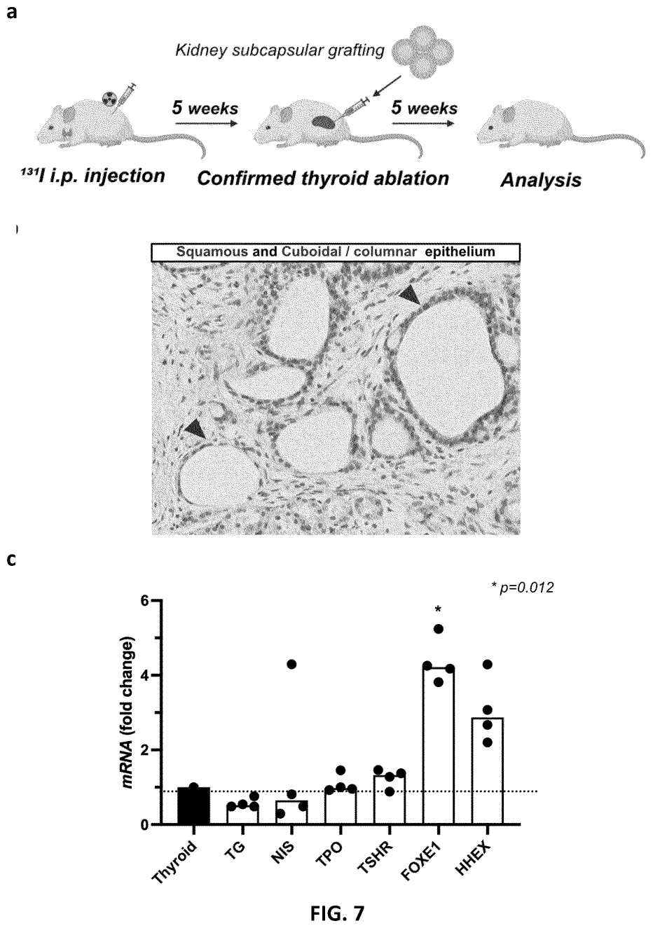

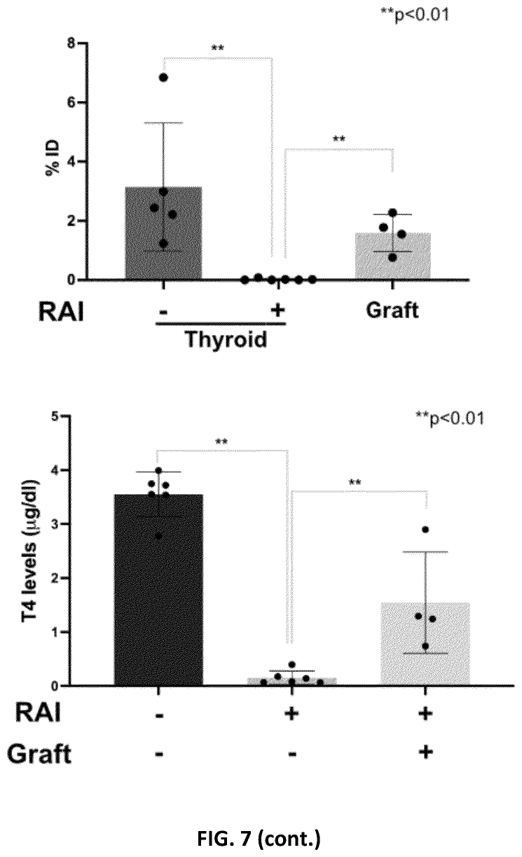

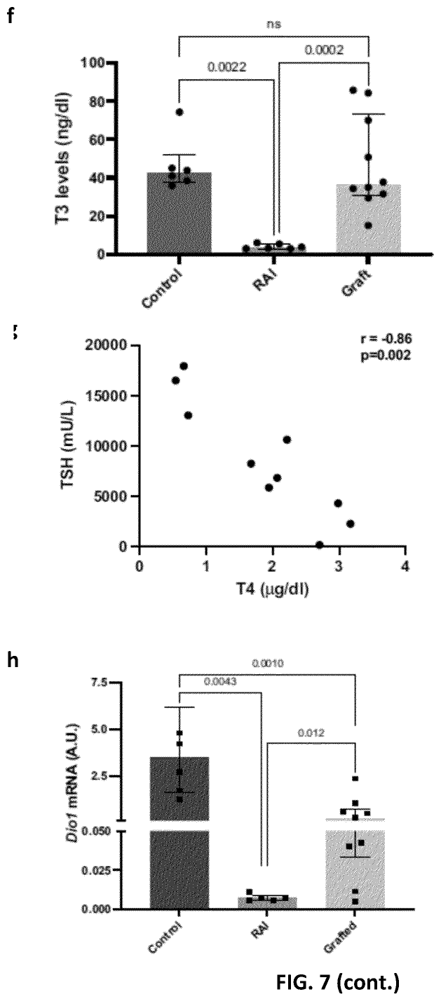

Organoids were harvested and filtered at day 45 to remove most isolated cells and transplanted under the kidney capsule of mice with intact and ablated thyroid glands (FIG. 7a). Due to technical problems caused by the radiosensitivity of immunodeficient mice, 60% of the irradiated animals died during the experimental period. At the end of the experiment, 6 non-transplanted and 4 transplanted mice had survived and could be compared with 6 untreated animals that served as controls.

Histological evaluation of the renal region five weeks after transplantation showed successful implantation of the transplanted organoids in the host niche (data not shown). HE staining showed numerous follicles organized in a manner characteristic of thyroid tissue (data not shown).

Blood vessels and stromal cells were observed in the vicinity of the thyroid follicles (data not shown). The presence of blood vessels in close proximity to the thyroid follicles is essential for the TH release and transport to target tissues. Immunostaining for the platelet-derived endothelial cell adhesion molecule CD31 revealed a dense network of small blood vessels surrounding the thyroid follicles, demonstrating the formation of classic angio-follicular units (data not shown). The absence of staining overlap between CD31 and Human Nuclear Antigen (HNA) provided unequivocal evidence that the vessels originated from host cells (data not shown). On the other hand, the stromal cells were derived from the grafted cells since they co-expressed HNA and a-SMA (data not shown).

HE staining showed that the derived follicular epithelium included both active follicles, which appeared cuboidal to low columnar, and inactive ones, in which the cells were squamous (FIG. 7b). Further immunohistochemical analysis supported the formation of functional thyroid follicles (NKX2-1+) at the graft site, including cell polarization labelled by E-Cadherin, TG cytosolic expression and deposition in the luminal compartment, and the appearance of TPO in the cytoplasm and mainly at the apical membrane (data not shown).

The transplanted tissue had a similar thyroid gene expression signature compared to human thyroid tissue (FIG. 7c).

SPECT-CT imaging was used to track the human thyroid graft performance, by the ability of NIS-dependent iodide uptake by thyroid tissue as described in Brandt et al. (2012). Images were acquired four weeks after transplantation and showed a strong uptake signal in the neck (where the thyroid gland is located) of non-ablated mice. In thyroid gland ablated transplanted mice, ¹²³I uptake was markedly decreased in the neck, but a very strong signal was detectable at the site of transplantation, near the kidney (data not shown). ¹²³I quantification in SPECT images expressed as percent injected dose (% ID) confirmed the uptake capacity of the transplanted tissue, with a % ID slightly lower compared to the thyroid tissue, while very low uptake values were detected in the neck of the hypothyroid non-transplanted mice (1.66 (0.96-2.14); 2.40 (1.72-4.91) and 0.010 (0.0023-0.032) % ID, respectively; p<0.01; FIG. 7d).

More importantly, transplanted animals presented a marked increase in plasma T4 levels (1.26 (0.86-2.49) μg/dl) compared to barely detectable plasma T4 levels in non-transplanted animals (0.11 (0.06-0.23) μg/dl), however still lower than controls non-irradiated (3.63 (3.35-3.80) (FIG. 7e). Evidence of functionality of the transplanted tissue was also provided by immunostaining for T4, which showed numerous active follicles with strong T4 signal in the luminal compartment (data not shown).

Interestingly, T3 levels were induced among grafted mice at levels similar to those of the control group, while they were significantly reduced in RAI-ablated mice (36.40 (31.08-73.68), 42.45 (37.85-52.35) and 3.50 (3.00-5.55) μg/dl; respectively; FIG. 7f).

Also the systemic impact of the thyroid organoid grafts was evaluated. Along with the restoration of THs plasma levels, grafted mice also showed a decrease in plasma TSH levels, which was inversely correlated to T4 (r=−0.86; FIG. 7g).

Studies have shown that modulation of thyroid status leads to changes in type 1 deiodinase (Dio1; D1) levels in the liver. To assess systemic recovery, Dio1 mRNA levels were measured in the liver of the grafted, RAI-ablated and control mice. Notably, hypothyroid mice (RAI) had significantly lower levels than controls (0.0069 (0.0056-0.0090) A.U. and 3.471 (1.58-6.14) A.U., respectively), which was partially offset by transplantation of hESC-derived thyroid follicles (0.177 (0.032-0.709) A.U.; FIG. 7h). In addition, Dio1 mRNA levels strongly correlated with T4 and T3 levels (r=0.84 and r=0.71, respectively).

In conclusion, human functional thyroid tissue generated from pluripotent stem cells can be grafted and keep the functionality in vivo, producing THs and increasing T4 and T3 levels in hypothyroid animals.

### Example 5: In Vitro Differentiation of Human Lung Tissue

For the generation of in vitro lung tissue, the modified hESCs as described in Example 2 were used. For cell culture and expansion, modified hES cells were cultured on Matrigel-coated 6-multiwell plates using Stem Flex medium (Thermo Scientific, A3349401) and the medium was changed every day.

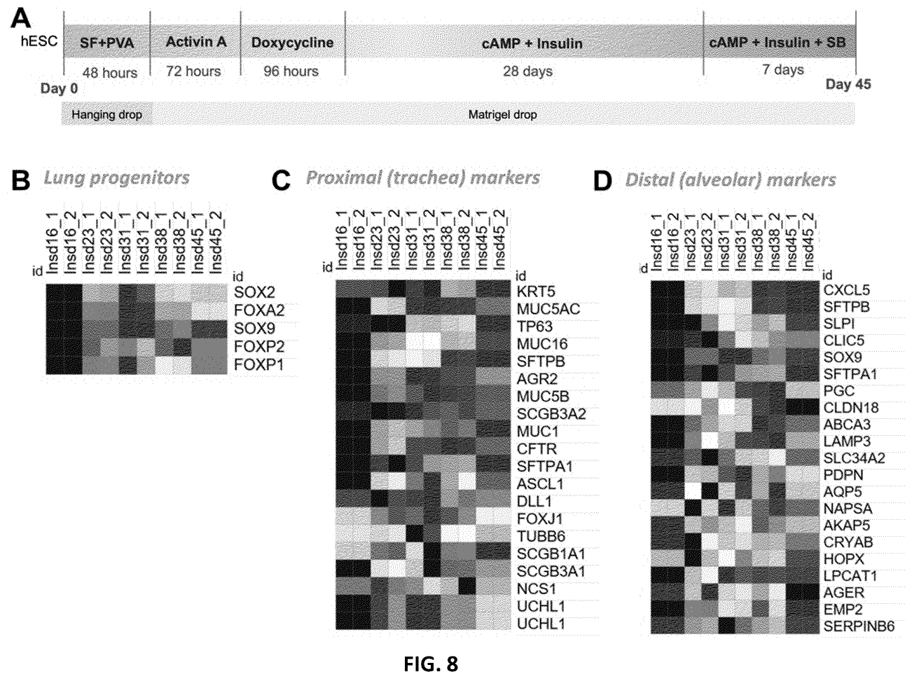

The method for generating lung organoids has been schematically presented in FIG. 8. Embryoid bodies were prepared as described in Examples 1 and 2 by hanging drop culture. At day 2, embryoid bodies were collected and resuspended in basal differentiation medium. The embryoid bodies were then cultured in Matrigel drops and endoderm was induced by adding Activin A for 3 days as described in Examples 1 and 2. After induction of the endoderm, at day 5, Doxycyline treatment promoted the overexpression of NKX2-1 and PAX8 as described in Examples 1 and 2. From day 9 to day 37 organoids were incubated with basal differentiation medium containing 0.3 nmol of 8-Br-cAMP (BioLog, B007-100) and 5 ug/ml of Insulin (Roche, 1137647001) to allow lung progenitors expansion and differentiation. From day 37 until day 45, organoids were cultured with the previously described differentiation medium containing 8-Br-cAMP and Insulin, and TGF-beta inhibitor SB431542 was added at a final concentration of 10 μM (Peprotech, 3014193), which promoted final maturation of the alveoli cells.

### Example 6: Characterization of Human Lung Organoids

For the characterization of the human lung organoids, the protocols for bulk RNA sequencing, qRT-PCR analysis and immunofluorescence analysis as described in Example 1 were used.

TABLE 4
Primer sequences used for qRT-PCR analysis of lung organoids.

| | Primer Forward | | Primer Reverse | |
|---|---|---|---|---|
| | | SEQ ID | | SEQ ID |
| Gene name | Sequence | NO: | Sequence | NO: |
| TP63 | CTCGTCCACCAGTCCCTATAA | 27 | TGATGGAGAGAGAGCATCGAA | 28 |
| FOXJ1 | GGCATAAGCGCAAACAGCCG | 29 | TCGAAGATGGCCTCCCAGTCAAA | 30 |
| SCGB3a2 | GAGGGGCTAAGGAAGTGTGT | 31 | CCATCCACCTCCGCTCTTTA | 32 |
| AGER | GCCACTGGTGCTGAAGTGTA | 33 | GTCCGGCCTGTGTTCAGTTT | 34 |
| LAMP3 | TGACCACTTTGGAAATGTGGATGAG | 35 | GATGGCCCCAATCACAGGAA | 36 |

TABLE 5
Primary antibodies used for immunofluorescence
staining of lung organoids.

| Protein | | Catalog | Host | Dilution |
|---|---|---|---|---|
| target | Supplier | number | Species | IF |
| E-cadherin | BD Biosciences | 610181 | Mouse | 1:500 |
| Mucin5a5 | Abcam | ab3649 | Mouse | 1:100 |
| P63 | Abcam | ab735 | Mouse | 1:50 |
| Keratin 5 | Cell signaling | 25807S | Rabbit | 1:500 |
| SOX2 | R&D Systems | AF2018 | Goat | 1:100 |
| HOPX | Proteintech | 11419-1-AP | Rabbit | 1:250 |
| NKX2.1 | Abcam | ab76013 | Rabbit | 1:500 |
| Ac Tubulin | Invitrogen | MA5-33079 | Rabbit | 1:200 |

### Scanning Electron Microscopy

Lung organoids at day 45 embedded in Matrigel were fixed using glutaraldehyde 2.5% overnight at 4° C., rinsed, and embedded in agarose 4%. Sections of 300 μm were produced in a Vibratome (Leica) and post-fixed in OsO4 (2%) for 1 hour. All treatments were done in 0.1 M cacodylate buffer (pH 7.2). After serial dehydration in ethanol, samples were dried at critical point and coated with platinum by standard procedures. Observations were done in a Tecnai FEG ESEM QUANTA 200 (FEI) at 30 kV and images were acquired and processed by SIS iTEM software (Olympus).

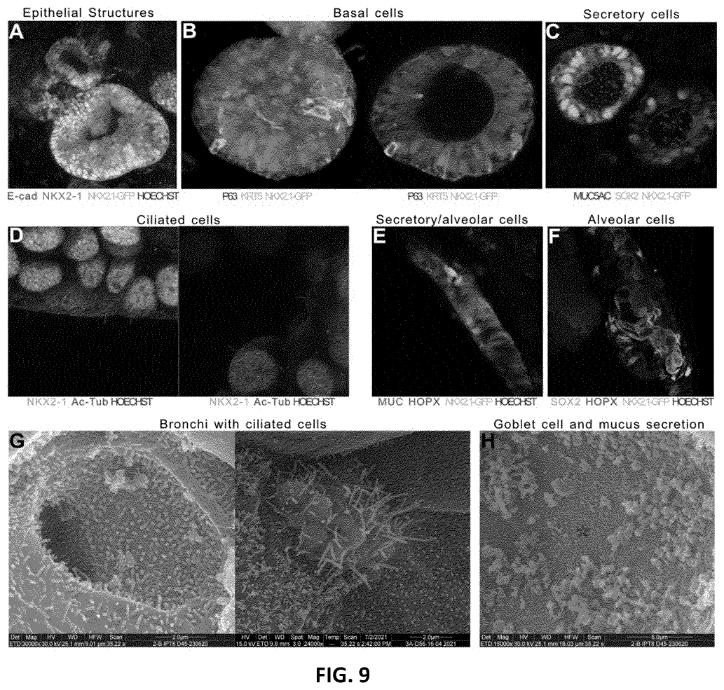

During the human lung differentiation protocol from hESC as schematically shown in FIG. 8A, lung progenitor markers were more prominently expressed between day 23 and day 31 (FIG. 8B), while markers of airway proximal cells, such as basal, secretory, ciliated and neuroendocrine cells (FIG. 8C) were mainly expressed from day 31 and alveolar markers (AT1 and AT2) appeared from day 38 with overall higher levels at day 45 (FIG. 8D). At day 45, lung structures were obtained, which contained basal cells (FIG. 9B), secretory cells (FIG. 9C), ciliated cells (FIG. 9D) and alveolar cells (FIG. 9F).

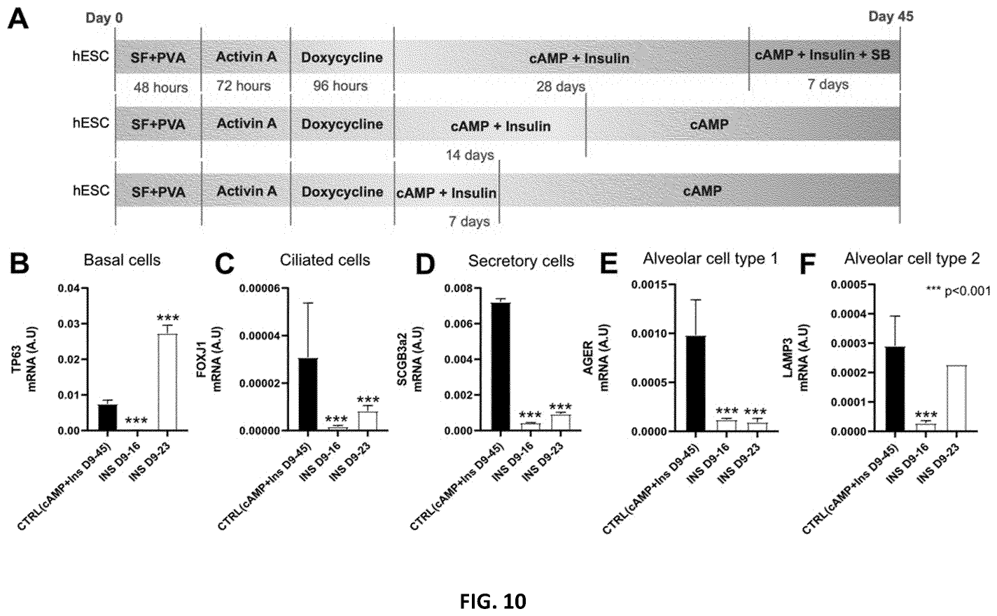

Insulin treatment for only one week (day 9-16) strongly reduced the expression of markers for all lung cell types (FIG. 10 B-F) while incubation with insulin for 2 weeks (day 9-23) appeared to induce immature lung structures evidenced by TP63 high levels (FIG. 10 B) while the other markers were significantly downregulated (FIG. 10 C-F).

1. A (in vitro) method for generating a thyroid organoid or a lung organoid from human pluripotent stem cells, said method comprising:
a. generating an embryoid body (EB) from human pluripotent stem cells (hPSCs);
b. culturing the embryoid body in a 3D matrix under conditions to induce differentiation into definitive endoderm;
c. transiently overexpressing NKX2-1 and PAX8 in the definitive endoderm to induce differentiation of endodermal cells into thyroid and lung progenitor cells; and
d. culturing the thyroid and lung progenitor cells in the 3D matrix in a differentiation medium for a period of time sufficient to generate a thyroid organoid or a lung organoid.

2. The method according to claim 1, wherein said human pluripotent stem cells are human embryonic stem cells.

3. (canceled)

4. The method according to claim 1, wherein inducing differentiation of the embryoid body into definitive endoderm (step b) comprises contacting the embryoid body in the 3D matrix with Activin A for about 1 to about 5 days.

5-7. (canceled)

8. The method according to claim 1, wherein the human pluripotent stem cells have been genetically modified with an inducible expression system encoding NKX2-1 and PAX8 and wherein transient NKX2-1 and PAX8 overexpression in the endodermal cells (step c) comprises culturing the definitive endoderm in the 3D matrix in a basal differentiation medium supplemented with an agent that activates the inducible expression system.

9. (canceled)

10. The method according to claim 1, wherein NKX2-1 and PAX8 are overexpressed during maximal 10 days.

11. (canceled)

12. The method according to claim 1 for generating a thyroid organoid, wherein culturing the thyroid and lung progenitor cells (step d) comprises culturing in a first differentiation medium comprising a basal differentiation medium and a cyclic monophosphate (cAMP), and subsequent culturing in a second differentiation medium comprising the basal differentiation medium and a glucocorticoid and a thyroid-stimulating hormone.

13. The method according to claim 12, wherein a TGF-beta inhibitor is added to the second differentiation medium after about 7 days of culture in said second differentiation medium.

14. (canceled)

15. (canceled)

16. The method according to claim 12, wherein the thyroid and lung progenitor cells are cultured in the first differentiation medium for about 14 days and in the second differentiation medium for at least about 28 days

17. The method according to claim 12, wherein step d further comprises culturing the thyroid and lung progenitor cells in the basal differentiation medium for between about 5 and about 9 days before culturing them in the first differentiation medium.

18. The method according to claim 1 for generating a lung organoid, wherein culturing the thyroid and lung progenitor cells (step d) comprises culturing in a differentiation medium comprising a basal differentiation medium, a cyclic monophosphate (cAMP), and insulin or insulin-like growth factor 1 (IGF-1).

19. The method according to claim 18, wherein a TGF-beta inhibitor is added to the differentiation medium after about 28 days of culture in the differentiation medium.

20. The method according to claim 18, wherein the thyroid and lung progenitor cells are cultured in the differentiation medium for at least about 36 days.

21. (canceled)

22. A thyroid organoid obtainable by the method according to claim 1.

23-26. (canceled)

27. A method of preventing or treating a thyroid disease or disorder, said method comprising implanting the thyroid organoid according to claim 22 to a subject in need thereof.

28. (canceled)

29. A method for modeling thyroid development or for modelling a thyroid disease or disorder or for screening for drugs for the treatment of a thyroid disease or disorder using the thyroid organoid according to claim 22.

30-32. (canceled)

33. A method for studying the effect of a test compound on thyroid tissue, said method comprising:
contacting the test compound with the thyroid organoid according to claim 22, and
determining the effect of the test compound on the thyroid organoid.

34. A lung organoid obtainable by the method according to claim 1.

35-41. (canceled)

42. A method for modelling lung development or for modelling a lung disease or disorder or for screening for drugs for the treatment of a lung disease or disorder using the lung organoid according to claim 34.

43-45. (canceled)

46. A method for studying the effect of a test compound on lung tissue, said method comprising:
contacting the test compound with the lung organoid according to claim 34, and
determining the effect of the test compound on the lung organoid.

47. (canceled)

48. A method of preventing or treating lung disease or disorder, said method comprising implanting the lung organoid according to claim 34 to a subject in need thereof.
